(19) United States
(12) Patent Application Publication
(10) Pub. No.: US 2014/0121233 A1
(43) Pub. Date: May 1, 2014

(54) Synthesis and Characterization of Second Generation Benzofuranone Ring Substituted Noscapine Analogs

(71) Applicants: Ritu Aneja, Lilburn, GA (US); Ram Chandra Mishra, Athens, GA (US)

(72) Inventors: Ritu Aneja, Lilburn, GA (US); Ram Chandra Mishra, Athens, GA (US)

(73) Assignee: GEORGIA STATE UNIVERSITY RESEARCH FOUNDATION, INC., Atlanta, GA (US)

(21) Appl. No.: 14/124,169

(22) PCT Filed: Jun. 11, 2012
(86) PCT No.: PCT/US2012/041898

§ 371 Date: Dec. 5, 2013

Related U.S. Application Data
(60) Provisional application No. 61/495,564, filed on Jun. 10, 2011.

(51) Int. Cl. C07D 491/056

(52) U.S. Cl. CPC C07D 491/056
USPC 514/291; 546/90

(57) ABSTRACT

Compound of Formula (I) and use thereof as microtubule modulating agents in the treatment of cancer are described herein.

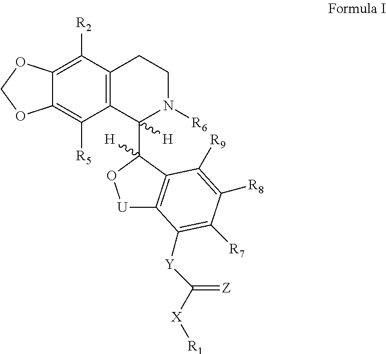

### STATEMENT REGARDING FEDERALLY SPONSORED RESEARCH OR DEVELOPMENT

This invention was made with Government Support under Agreement R00CA131489 awarded to Ritu Aneja by the National Cancer Institute. The Government has certain rights in the invention.

### FIELD OF THE INVENTION

This invention is generally in the field of noscapine analogs, particularly noscapine substituted at the 7-position of the isobenzofuranone ring, pharmaceutical compositions containing the analogs, and methods of making and using thereof.

### BACKGROUND OF THE INVENTION

Microtubules are composed of α/β tubulin heterodimers. Microtubules are ubiquitous dynamic cytoskeletal polymers that have been long recognized as a pharmaceutical target in cancer chemotherapy. Drugs that interfere with microtubule dynamic stability have been employed in the clinic to treat a variety of cancers or are exploited as probes to gain insights into microtubule structure and function. Three major classes of drugs, taxanes, vinca alkaloids and colchicine analogs, are known in the art and the positions they occupy on the cellular target, tubulin, have been identified. Traditionally, these three drug classes are categorized into stabilizers and destabilizers; the stabilizers predominantly causing overpolymerization of microtubules into bundles and sheets and the destabilizers resulting in depolymerization of microtubules into soluble tubulin.

Yet another emerging class of microtubule-modulating agents is based upon noscapine, a non-sedative naturally-occurring phthalideisoquinoline alkaloid from the opium poppy. Noscapine has been shown to exhibit tubulin-binding anticancer activity. Specifically, noscapine has been shown to inhibit various neoplasms in vitro as well as in vivo such as leukemia and lymphoma, melanoma, ovarian, gliomas, breast, lung, and colon cancer. Currently, noscapine is in Phase I/II clinical trials for the treatment of multiple myeloma.

Ongoing chemical synthetic efforts to improve the therapeutic efficacy and pharmacological properties of noscapine have yielded a battery of more potent first-generation noscapine analogs, collectively referred to as noscapinoids. Noscapinoids may avoid the harsher effects of currently-available chemotherapeutic agents by leaving the total polymer mass of tubulin unaffected. Noscapine analogs have been shown to impede cell-cycle progression, inhibit cellular proliferation and induce apoptosis in a variety of cancer cells both in vitro and in xenograft models of human cancers implanted in nude mice. From a synthetic perspective, the majority of these first-generation analogs were generated by the chemical manipulation of position-9 on the isoquinoline ring system of noscapine (FIG. 1). Specific analogs that have been evaluated include 9-nitronoscapine as well as halogenated (fluoro, chloro, bromo, and iodo) analogs. In particular, the brominated analog of noscapine has been studied extensively because of its effectiveness against drug-resistant xenograft tumors without any detectable toxicity. However, less work has been done regarding noscapinoids derivatized at positions other than the 9-position.

There exists a need for novel analogs of noscapine that are as effective or are more effective that noscapine and exhibit reduced toxicity. There also exists a need to for formulations of these analogs in order to improve the solubility and bioavailability of the compounds.

Therefore, it is an object of the invention to provide analogs of noscapine that are as effective or are more effective that noscapine and exhibit reduced toxicity and methods of making and using thereof.

It is also an object of the invention to provide formulations having improved solubility and bioavailability of the noscapine analogs described herein.

### SUMMARY OF THE INVENTION

Compounds of Formula I are described herein:

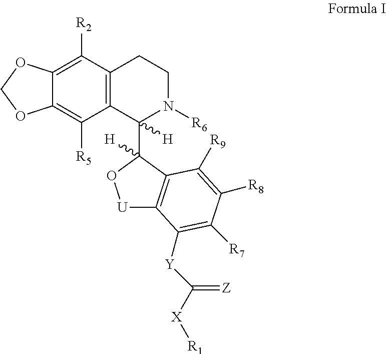

wherein,

R₁ is selected from hydrogen; substituted or unsubstituted alkyl, alkenyl, alkynyl, cycloalkyl, heterocycloalkyl, cycloalkenyl, heterocycloalkenyl, aryl, and heteroaryl;

X and Y are independently absent or S, O, and NR₃, wherein R₃ is H, alkyl, substituted or unsubstituted alkyl, alkenyl, alkynyl, cycloalkyl, heterocycloalkyl, cycloalkenyl, heterocycloalkenyl, aryl, and heteroaryl;

Z is O, S, or N;

R₂, R₅, and R₇-R₉ are independently selected from hydrogen; halogen, substituted or unsubstituted alkyl, alkenyl, alkynyl, cycloalkyl, heterocycloalkyl, cycloalkenyl, heterocycloalkenyl, aryl, heteroaryl, alkylaryl, alkylheteroaryl, arylalkyl, or heteroarylalkyl; —OR′; —NR′R″; —(CH₂)ₘNR′R″, wherein m is 0, 1, or 2; —NO₂; —CF₃; —CN; —C₂R′; —SR′; —N₃; —C(═O)ₘNR′R″; —NR′C(═O)R″; —C(═O)R′; —C(═O)OR′; —OC(═O)R′; —O(CR′R″)ᵣC(═O)R′; —O(CR′R″)ᵣNR″C(═O)R′; —O(CR′R″)ᵣNR″SO₂R′; —OC(═O)NR′R″; —NR′C(═O)OR″; —SO₂R′; —SO₂NR′R″; and —NR′SO₂R″; wherein R′ and R″ are individually hydrogen or substituted or unsubstituted alkyl, alkenyl, alkynyl, cycloalkyl, heterocyclyl, cycloalkenyl, heterocycloalkenyl, aryl, heteroaryl, arylalkyl, heteroalkyls, alkylaryl, alkylheteroaryl, and r is an integer from 1 to 6;

R₆ is hydrogen or substituted or unsubstituted alkyl, alkenyl, alkynyl, cycloalkyl, heterocycloalkyl, cycloalkenyl, heterocycloalkenyl, aryl, heteroaryl, arylalkyl, heteroarylalkyl, alkylaryl, alkylheteroaryl, —C(═W)NR′R″, wherein W is O, S, or N and R′ and R″ are as defined above; and

U is CH₂; C═O; C═S; C═NH, C═NR, wherein R is as defined above for R′ and R″; CHOH, CHOR, wherein R is as defined above for R′ and R″; or CR₁₀R₁₁, wherein R₁₀ and R₁₁ are as defined above for R₂.

In some embodiments, R₆ is —C(═W)NR′R″, wherein W is O or S, R′ is hydrogen, and R″ is 3-chlorophenyl, 4-chlorophenyl, 2,4-dichlorophenyl, 2,4-difluorophenyl, phenyl, 2-methoxyphenyl, 4-methylphenyl, or 1-naphthyl and R is hydrogen or bromine.

In other embodiments, Y is hydrogen; C₁₋₆ alkyl; C₁₋₆ alkylaryl; —C(═O)alkyl or —C(═O)C₁₋₆ alkylaryl; —CH₂—CH(OH)—CH₂T, where T is C₁₋₆ alkyl or —O—C₁₋₆ alkyl; aryl; or heteroaryl.

In still another embodiment, the compounds are of Formula II:

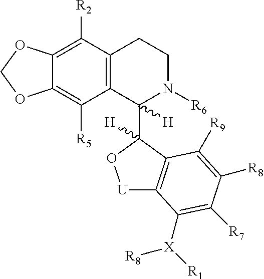

wherein R₂, R₅-R₉, and U are as defined above, X is N, S, or O, and R₁ and/or R₈ are absent or are independently selected from, as valence allows, hydrogen; substituted or unsubstituted alkyl, alkenyl, alkynyl, cycloalkyl, heterocycloalkyl, cycloalkenyl, heterocycloalkenyl, aryl, and heteroaryl.

In some embodiments, U is a group other than C═O. In other embodiments, U is C═O, R₂ is other than hydrogen or bromine, R₅ is other than methoxy, and/or R₆ is other than methyl.

In some embodiments, X is oxygen, R₈ is absent, and R₁ is 3,4,5-trimethoxy; O-3-thiophene; O-2-thiophene; O-3-thiophene; O-4-thiazole; 3,4-dimethoxy; 3,4-methylenedioxy-5-methoxy; or 3,4,5-triethoxy. In other embodiments, X is nitrogen, R₈ is hydrogen, and R₁ is 3,4,5-trimethoxy; O-3-thiophene; O-2-thiophene; O-3-thiophene; O-4-thiazole; 3,4-dimethoxy; 3,4-methylenedioxy-5-methoxy; or 3,4,5-triethoxy.

The compounds described herein can be combined with one or more pharmaceutically acceptable excipients to prepare pharmaceutical compositions. The compositions can be formulated for parenteral, enteral, topical, or pulmonary delivery. Suitable oral dosage forms include, but are not limited to, tablets, caplets, capsules, syrups, solutions, suspensions, and emulsions. Suitable injectable formulations include solutions and suspensions. Suitable topical formulations include lotions, creams, ointments, and patches. Suitable pulmonary formulations include solution, suspensions, or aerosols which can be inhaled into the lung.

The compounds can be administered alone or co-administered with one or more additional active agents, such as therapeutic, diagnostic, and/or prophylactic agents. Suitable classes of additional active agents include, but are not limited to, alkylating agents, such as ethylenimines and methylmelamines, alkyl sulfonates, and triazines; antimetabolites, such as folic acid and analogs thereof, pyrimidine analogs, and purine analogs and related inhibitors; cytotoxic anticancer agents, such as paclitaxel; cytostatic and/or cytotoxic agents including anti-angiogenic agents such as endostatin, angiostatin, and thalidomide; analgesics, such as opioid and non-opioid analgesics; vaccines containing cancer antigens or immunomodulators such as cytokines to enhance the anti-cancer activity; natural products, such as vinca alkaloids, epipodophyllotoxins, antibiotics, enzymes, and biological response modifiers; hormones and antagonists, such as adrenocorticosteroids, progestins, estrogens, antiestrogen, androgens, and gonadotropin-releasing hormone analogs; and miscellaneous compounds, such as platinum coordination complexes, anthracenedione, substituted urea, methyl hydrazines, and adrenocortical suppressants.

The compounds described herein can be used to treat a variety of diseases or disorders. Exemplary disorders include proliferative disorders, such as cancer and hypoxic ischemia.

### BRIEF DESCRIPTION OF THE DRAWINGS

FIG. 1 is the chemical structure of noscapine showing the numbering system for identifying atom position in the dioxoloisoquinoline ring and the isobenzofuranone ring.

FIG. 2A is the chemical structure of colchine. FIG. 2B is the chemical structure of noscapine. FIG. 2C is a molecular modeling representation showing the overlap of the chemical structures of colchine and noscapine. The geometric complementarity score is 72.75%.

FIGS. 3A-3E are line graphs showing the inhibition of tubulin assembly (absorbance) by noscapine analogs 3-7 in vitro as a function of time (minutes). FIG. 3F is a line graph showing the percent of tubulin polymerization as a function of concentration (μg/ml) for analogs 3-7.

FIGS. 4A-E are line graphs showing the anti-proliferative activity (percent survival) of compounds 3-7 as a function of concentration (μM) in various cancer cell lines using the MTT assay. FIG. 4F is a bar graph showing the IC₅₀ value for compounds 3-7 for the cell lines A549, CEM, MCF-7, MIA PaCa-2, and PC-3.

FIGS. 5A-E are line graphs showing the activity of compounds 3-7 against certain cell lines (percent survival) as a function of concentration (μM) of the analog. FIG. 5F is a bar graph showing the IC₅₀ values of noscapine analogs in the various cancer cell lines.

FIGS. 6Ai-Ei are cell-cycle distributions of MDA-MB-231 cells in a three-dimensional disposition as determined by flow cytometry at different time points upon treatment with 25 μM of analogs 3-7. FIGS. 6Aii-Eii are bar graphs showing the percent G2/M and sub-G1 populations at different time points for analogs 3-7.

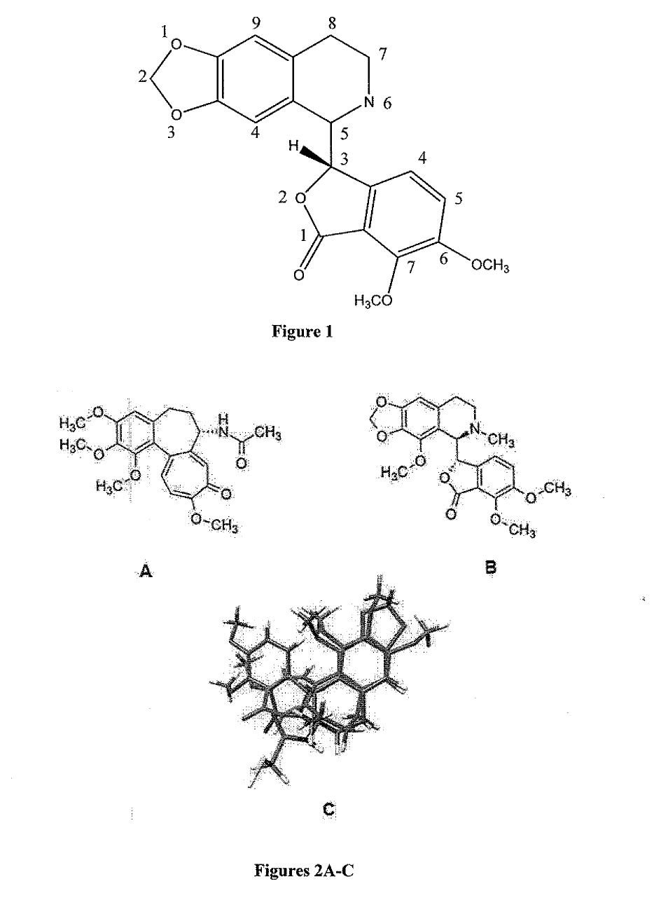

### DETAILED DESCRIPTION OF THE INVENTION

### I. Definitions

The “effective amount”, e.g., of the noscapine analogs described herein, refers to an amount of the analog in a composition or formulation which, when applied as part of a desired dosage regimen brings about, e.g., a change in the rate of cell proliferation and/or the state of differentiation of a cell and/or rate of survival of a cell according to clinically acceptable standards for the disorder to be treated.

The “growth state” of a cell refers to the rate of proliferation of the cell and/or the state of differentiation of the cell. An “altered growth state” is a growth state characterized by an abnormal rate of proliferation, e.g., a cell exhibiting an increased or decreased rate of proliferation relative to a normal cell.

The term “patient” or “subject” to be treated refers to either a human or non-human animal.

The term “prodrug”, as used herein, refers to compounds which, under physiological conditions, are converted into the therapeutically active agents of the present invention. A common method for making a prodrug is to include selected moieties which are hydrolyzed under physiological conditions to reveal the desired molecule. In other embodiments, the prodrug is converted by an enzymatic activity of the host animal.

As used herein, “proliferating” and “proliferation” refer to cells undergoing mitosis.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Stereoisomer”, as used herein, refers to isomeric molecules that have the same molecular formula and sequence of bonded atoms (constitution), but which differ in the three dimensional orientations of their atoms in space. Examples of stereoisomers include enantiomers and diastereomers. As used herein, an enantiomer refers to one of the two mirror-image forms of an optically active or chiral molecule. Diastereomers (or diastereoisomers) are stereoisomers that are not enantiomers (non-superimposable mirror images of each other). Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2n, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Enantiomers and/or diasteromers can be resolved or separated using techniques known in the art.

“Half maximal inhibitory concentration, IC₅₀”, as used herein, refers to a measure of the effectiveness of a compound in inhibiting biological or biochemical function. This quantitative measure indicates how much of a particular drug or other substance (inhibitor) is needed to inhibit a given biological process (or component of a process, i.e. an enzyme, cell, cell receptor or microorganism) by half. According to the FDA, IC₅₀ represents the concentration of a drug that is required for 50% inhibition in vitro. The IC₅₀ can be determined using a variety of assays known in the art.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups.

In preferred embodiments, a straight chain or branched chain alkyl has 30 or fewer carbon atoms in its backbone (e.g., C₁-C₃₀ for straight chains, C₃-C₃₀ for branched chains), preferably 20 or fewer, more preferably 15 or fewer, most preferably 10 or fewer. Likewise, preferred cycloalkyls have from 3-10 carbon atoms in their ring structure, and more preferably have 5, 6 or 7 carbons in the ring structure. The term “alkyl” (or “lower alkyl”) as used throughout the specification, examples, and claims is intended to include both “unsubstituted alkyls” and “substituted alkyls”, the latter of which refers to alkyl moieties having one or more substituents replacing a hydrogen on one or more carbons of the hydrocarbon backbone. Such substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, a hosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, or an aromatic or heteroaromatic moiety.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Throughout the application, preferred alkyl groups are lower alkyls. In preferred embodiments, a substituent designated herein as alkyl is a lower alkyl.

It will be understood by those skilled in the art that the moieties substituted on the hydrocarbon chain can themselves be substituted, if appropriate. For instance, the substituents of a substituted alkyl may include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Cycloalkyls can be substituted in the same manner.

The term “heteroalkyl”, as used herein, refers to straight or branched chain, or cyclic carbon-containing radicals, or combinations thereof, containing at least one heteroatom. Suitable heteroatoms include, but are not limited to, O, N, Si, P, Se, B, and S, wherein the phosphorous and sulfur atoms are optionally oxidized, and the nitrogen heteroatom is optionally quaternized. Heteroalkyls can be substituted as defined above for alkyl groups.

The term “alkylthio” refers to an alkyl group, as defined above, having a sulfur radical attached thereto. In preferred embodiments, the “alkylthio” moiety is represented by one of —S-alkyl, —S-alkenyl, and —S-alkynyl. Representative alkylthio groups include methylthio, ethylthio, and the like. The term “alkylthio” also encompasses cycloalkyl groups, alkene and cycloalkene groups, and alkyne groups. “Arylthio” refers to aryl or heteroaryl groups. Alkylthio groups can be substituted as defined above for alkyl groups.

The terms “alkenyl” and “alkynyl”, refer to unsaturated aliphatic groups analogous in length and possible substitution to the alkyls described above, but that contain at least one double or triple bond respectively.

The terms “alkoxyl” or “alkoxy” as used herein refers to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, and —O-alkynyl. Aroxy can be represented by —O-aryl or O-heteroaryl, wherein aryl and heteroaryl are as defined below. The alkoxy and aroxy groups can be substituted as described above for alkyl.

The terms “amine” and “amino” are art-recognized and refer to both unsubstituted and substituted amines, e.g., a moiety that can be represented by the general formula:

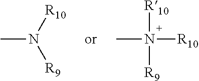

wherein R₉, R₁₀, and R′₁₀ each independently represent a hydrogen, an alkyl, an alkenyl, —(CH₂)ₘ—R₈ or R₉ and R₁₀ taken together with the N atom to which they are attached complete a heterocycle having from 4 to 8 atoms in the ring structure; R₈ represents an aryl, a cycloalkyl, a cycloalkenyl, a heterocycle or a polycycle; and m is zero or an integer in the range of 1 to 8. In preferred embodiments, only one of R₉ or R₁₀ can be a carbonyl, e.g., R₉, R₁₀ and the nitrogen together do not form an imide. In still more preferred embodiments, the term “amine” does not encompass amides, e.g., wherein one of R₉ and R₁₀ represents a carbonyl. In even more preferred embodiments, R₉ and R₁₀ (and optionally R′₁₀) each independently represent a hydrogen, an alkyl or cycloakly, an alkenyl or cycloalkenyl, or alkynyl. Thus, the term “alkylamine” as used herein means an amine group, as defined above, having a substituted (as described above for alkyl) or unsubstituted alkyl attached thereto, i.e., at least one of R₉ and R₁₀ is an alkyl group.

The term “amido” is art-recognized as an amino-substituted carbonyl and includes a moiety that can be represented by the general formula:

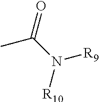

wherein R₉ and R₁₀ are as defined above.

“Aryl”, as used herein, refers to C₅-C₁₀-membered aromatic, heterocyclic, fused aromatic, fused heterocyclic, biaromatic, or bihetereocyclic ring systems. Broadly defined, “aryl”, as used herein, includes 5-, 6-, 7-, 8-, 9-, and 10-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with one or more substituents including, but not limited to, halogen, azide, alkyl, aralkyl, alkenyl, alkynyl, cycloalkyl, hydroxyl, alkoxyl, amino (or quaternized amino), nitro, sulfhydryl, imino, amido, phosphonate, phosphinate, carbonyl, carboxyl, silyl, ether, alkylthio, sulfonyl, sulfonamide, ketone, aldehyde, ester, heterocyclyl, aromatic or heteroaromatic moieties, —CF₃, —CN; and combinations thereof.

The term “aryl” also includes polycyclic ring systems having two or more cyclic rings in which two or more carbons are common to two adjoining rings (i.e., “fused rings”) wherein at least one of the rings is aromatic, e.g., the other cyclic ring or rings can be cycloalkyls, cycloalkenyls, cycloalkynyls, aryls and/or heterocycles. Examples of heterocyclic rings include, but are not limited to, benzimidazolyl, benzofuranyl, benzothiofuranyl, benzothiophenyl, benzoxazolyl, benzoxazolinyl, benzthiazolyl, benzthiazolyl, benztetrazolyl, benzisoxazolyl, benzisothiazolyl, benzimidazolinyl, carbazolyl, 4aH carbazolyl, carbolinyl, chromenyl, chromenyl, cinnolinyl, decahydroquinolinyl, 2H,6H-1,5,2-dithiazinyl, dihydrofuro[2,3b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined above for “aryl”.

The term “aralkyl”, as used herein, refers to an alkyl group substituted with an aryl group (e.g., an aromatic or heteroaromatic group).

The term “carbocycle”, as used herein, refers to an aromatic or non-aromatic ring in which each atom of the ring is carbon.

“Heterocycle” or “heterocyclic”, as used herein, refers to a cyclic radical attached via a ring carbon or nitrogen of a monocyclic or bicyclic ring containing 3-10 ring atoms, and preferably from 5-6 ring atoms, consisting of carbon and one to four heteroatoms each selected from the group consisting of non-peroxide oxygen, sulfur, and N(Y) wherein Y is absent or is H, O, (C₁-C₁₀) alkyl, phenyl or benzyl, and optionally containing 1-3 double bonds and optionally substituted with one or more substituents. Examples of heterocyclic ring include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxepanyl, oxetanyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydropyranyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. Heterocyclic groups can optionally be substituted with one or more substituents at one or more positions as defined above for alkyl and aryl, for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF3, —CN, or the like.

The term “carbonyl” is art-recognized and includes such moieties as can be represented by the general formula:

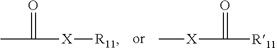

wherein X is a bond or represents an oxygen or a sulfur, and R₁₁ represents a hydrogen, an alkyl, a cycloalkyl, an alkenyl, an cycloalkenyl, or an alkynyl, R′₁₁ represents a hydrogen, an alkyl, a cycloalkyl, an alkenyl, an cycloalkenyl, or an alkynyl. Where X is an oxygen and R₁₁ or R′₁₁ is not hydrogen, the formula represents an “ester”. Where X is an oxygen and R₁₁ is as defined above, the moiety is referred to herein as a carboxyl group, and particularly when R₁₁ is a hydrogen, the formula represents a “carboxylic acid”. Where X is an oxygen and R′₁₁ is hydrogen, the formula represents a “formate”. In general, where the oxygen atom of the above formula is replaced by sulfur, the formula represents a “thiocarbonyl” group. Where X is a sulfur and R₁₁ or R′₁₁ is not hydrogen, the formula represents a “thioester.” Where X is a sulfur and R₁₁ is hydrogen, the formula represents a “thiocarboxylic acid.” Where X is a sulfur and R′₁₁ is hydrogen, the formula represents a “thioformate.” On the other hand, where X is a bond, and R₁₁ is not hydrogen, the above formula represents a “ketone” group. Where X is a bond, and R₁₁ is hydrogen, the above formula represents an “aldehyde” group.

The term “heteroatom” as used herein means an atom of any element other than carbon or hydrogen. Preferred heteroatoms are boron, nitrogen, oxygen, phosphorus, sulfur and selenium. Other heteroatoms include silicon and arsenic.

As used herein, the term “nitro” means —NO₂; the term “halogen” designates —F, —Cl, —Br or —I; the term “sulfhydryl” means —SH; the term “hydroxyl” means —OH; and the term “sulfonyl” means —SO₂—.

### II. Compounds

1. Noscapine Analogs

Compounds of Formula I are described herein:

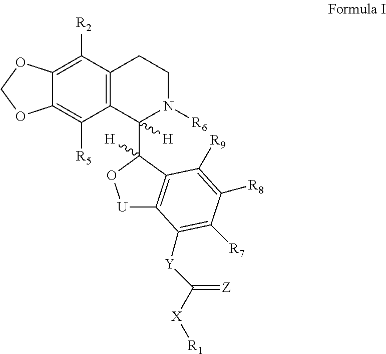

wherein,

R₁ is selected from hydrogen; substituted or unsubstituted alkyl, alkenyl, alkynyl, cycloalkyl, heterocycloalkyl, cycloalkenyl, heterocycloalkenyl, aryl, and heteroaryl;

X and Y are independently absent or S, O, and NR₃, wherein R₃ is H, alkyl, substituted or unsubstituted alkyl, alkenyl, alkynyl, cycloalkyl, heterocycloalkyl, cycloalkenyl, heterocycloalkenyl, aryl, and heteroaryl;

Z is O, S, or N;

R₂, R₅, and R₇-R₉ are independently selected from hydrogen; halogen, substituted or unsubstituted alkyl, alkenyl, alkynyl, cycloalkyl, heterocycloalkyl, cycloalkenyl, heterocycloalkenyl, aryl, heteroaryl, alkylaryl, alkylheteroaryl, arylalkyl, or heteroarylalkyl; —OR′; —NR′R″; —(CH₂)ₘNR′R″, wherein m is 0, 1, or 2; —NO₂; —CF₃; —CN; —C₂R′; —SR′; —N₃; —C(═O)NR′R″; —NR′C(═O)R″; —C(═O)R′; —C(═O)OR′; —OC(═O)R′; —O(CR′R″)ᵣC(═O)R′; —O(CR′R″)ᵣNR″C(═O)R′; —O(CR′R″)ᵣNR″SO₂R′; —OC(═O)NR′R″; —NR′C(═O)OR″; —SO₂R′; —SO₂NR′R″; and —NR′SO₂R″; wherein R′ and R″ are individually hydrogen or substituted or unsubstituted alkyl, alkenyl, alkynyl, cycloalkyl, heterocyclyl, cycloalkenyl, heterocycloalkenyl, aryl, heteroaryl, arylalkyl, heteroalkyls, alkylaryl, alkylheteroaryl, and r is an integer from 1 to 6;

R₆ is hydrogen or substituted or unsubstituted alkyl, alkenyl, alkynyl, cycloalkyl, heterocycloalkyl, cycloalkenyl, heterocycloalkenyl, aryl, heteroaryl, arylalkyl, heteroarylalkyl, alkylaryl, alkylheteroaryl, —C(═W)NR′R″, wherein W is O, S, or N and R′ and R″ are as defined above; and

U is CH₂; C═O; C═S; C═NH; C═NR, wherein R is as defined above for R′ and R″; CHOH or CHOR, wherein R is as defined above for R′ and R″; or CR₁₀R₁₁, wherein R₁₀ and R₁₁ are as defined above for R₂.

In some embodiments U is C═O. In some embodiments, U is C═O and R₅ is hydrogen or methoxy.

In some embodiments, U is C═O, R₅ is hydrogen or methoxy, and R₂ is hydrogen, bromine, or methoxy.

In some embodiments, U is C═O, R₅ is hydrogen or methoxy, R₂ is hydrogen, bromine, or methoxy, and R₆ is alkyl or aryl or —C(═W)NR′R″, wherein W is O, S, or N and R′ and R″ are as defined above.

In some embodiments, U is C═O, R₅ is hydrogen or methoxy, R₂ is hydrogen, bromine, or methoxy, R₆ is alkyl or aryl or —C(═W)NR′R″, wherein W is O, S, or N and R′ and R″ are as defined above, and R₈ and R₉ are hydrogen.

In some embodiments, U is C═O, R₅ is hydrogen or methoxy, R₂ is hydrogen, bromine, or methoxy, R₆ is alkyl or aryl or —C(═W)NR′R″, wherein W is O, S, or N and R′ and R″ are as defined above, R₈ and R₉ are hydrogen, and R₇ is methoxy.

In some embodiments, U is C═O, R₅ is hydrogen or methoxy, R₂ is hydrogen, bromine, or methoxy, R₆ is alkyl or aryl or —C(═W)NR′R″, wherein W is O, S, or N and R′ and R″ are as defined above, R₈ and R₉ are hydrogen, R₇ is methoxy, and Y—C(═Z)X—R₁ is as defined above.

In some embodiments, R₆ is —C(═W)NR′R″, wherein W is O or S, R′ is hydrogen, and R″ is 3-chlorophenyl, 4-chlorophenyl, 2,4-dichlorophenyl, 2,4-difluorophenyl, phenyl, 2-methoxyphenyl, 4-methylphenyl, or 1-naphthyl and R is hydrogen or bromine.

In other embodiments, Y is hydrogen; C₁₋₆ alkyl; C₁₋₆ alkylaryl; —C(═O)alkyl or —C(═O)C₁₋₆ alkylaryl; —CH₂—CH(OH)—CH₂T, where T is C₁₋₆ alkyl or —O—C₁₋₆ alkyl; aryl; or heteroaryl.

In still another embodiment, the compounds are of Formula II:

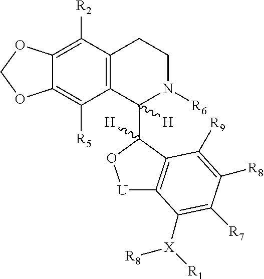

wherein R₂, R₅-R₉, and U are as defined above, X is N, S, or O, and R₁ and/or R₈ are absent or are independently selected from, as valence allows, hydrogen; substituted or unsubstituted alkyl, alkenyl, alkynyl, cycloalkyl, heterocycloalkyl, cycloalkenyl, heterocycloalkenyl, aryl, and heteroaryl.

In some embodiments, U is a group other than C═O. In other embodiments, U is C═O, R₂ is other than hydrogen or bromine, R₅ is other than methoxy, and/or R₆ is other than methyl.

In some embodiments, X is oxygen, R₈ is absent, and R₁ is 3,4,5-trimethoxy; O-3-thiophene; O-2-thiophene; O-3-thiophene; O-4-thiazole; 3,4-dimethoxy; 3,4-methylenedioxy-5-methoxy; or 3,4,5-triethoxy. In other embodiments, X is nitrogen, R₈ is hydrogen, and R₁ is 3,4,5-trimethoxy; O-3-thiophene; O-2-thiophene; O-3-thiophene; O-4-thiazole; 3,4-dimethoxy; 3,4-methylenedioxy-5-methoxy; or 3,4,5-triethoxy.

In other embodiment, X═S, R₁ is absent, and R₈ is as defined above. In specific embodiments, R₁ is benzyl.

In still other embodiment, X is nitrogen and R₁ and R₈ are as defined above. In specific embodiments, R₁ is benzyl, hydrogen, or methyl and R₈ is hydrogen.

The compounds of Formula I have at least one chiral center and therefore can exist as the following stereoisomers:

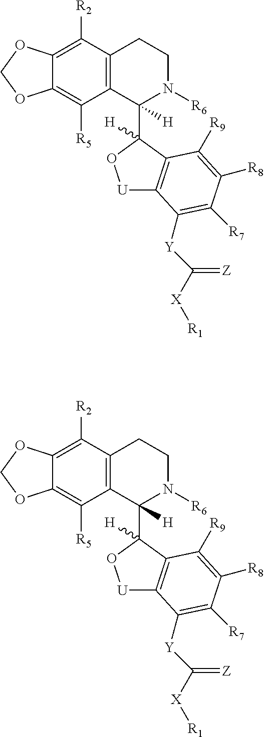

Similarly, Formula II can exist in the following forms:

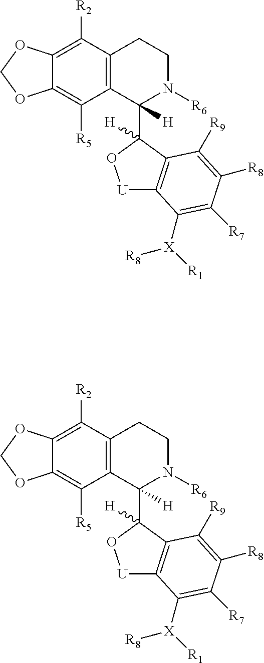

2. Noscapine Analog Conjugates

The noscapine analogs described herein may be linked to another molecule or molecules in order to improve the efficacy of the noscapine analogs. Suitable molecules include, but are not limited to, targeting agents and agents which increase the in vivo half life of the noscapine analogs (e.g., polyethylene glycol). The noscapine analogs can be linked to such molecules in any manner provided that each region of the conjugate continues to perform its intended function without significant impairment of biological activity, for example, the anti-tumor activity and/or anti-inflammatory activity of the compounds disclosed herein.

The noscapine analogs described herein may be directly linked to a second compound or may be linked via a linker. The term “linker”, as used herein, refers to one or more polyfunctional, e.g., bifunctional molecules, which can be used to covalently couple the one or more noscapine analogs to the molecule(s) and which do not interfere with the biological activity of the noscapine analogs. The linker may be attached to any part of the noscapine analogs so long as the point of attachment does not interfere with the biological activity, for example, the anti-tumor and/or anti-inflammatory activity of the compounds described herein.

In one embodiment, the noscapine analogs are conjugated to a second molecule through a reactive functional group on the noscapine analog, such as an ester, followed by reaction of the ester with a nucleophilic functional group on the molecule to be linked. The esters may be prepared, for example, by reaction of a carboxyl group on the noscapine analog with an alcohol in the presence of a dehydration agent such as dicyclohexylcarbodiimide (DCC), 1-(3-dimethylaminopropyl)-3-ethylcarbodiimide (EDC), or 1-(3-dimethylamino propyl)-3-ethylcarbodiimide methiodide (EDCI). The agent to be linked to the noscapine analog(s), for example, a tumor-specific antibody, is then mixed with the activated ester in aqueous solution to form the conjugate.

Alternatively, the ester of the noscapine analogs(s) may be prepared as described above and reacted with a linker group, for example, 2-aminoethanol, an alkylene diamine, an amino acid such as glycine, or a carboxy-protected amino acid such as glycine tert-butyl ester. If the linker contains a protected carboxy group, the protecting group is removed and the ester of the linker is prepared (as described above). The active ester is then reacted with the second molecule to give the conjugate. In another embodiment, the second agent can be derivatized with succinic anhydride to give an agent-succinate conjugate which may be condensed in the presence of EDC or EDCI with a linker having a free amino or hydroxyl group.

It also is possible to prepare a noscapine analog containing a linker with a free amino group and crosslink the free amino group with a heterobifunctional cross linker such as sulfosuccinimidyl 4-(N-maleimidocyclohexane)-1-carboxylate which will react with the free sulfhydryl groups of protein antigens.

The noscapine analogs may also be coupled to a linker by reaction of the aldehyde group with an amino linker to form an intermediate imine conjugate, followed by reduction with sodium borohydride or sodium cyanoborohydride. Examples of such linkers include amino alcohols such as 2-aminoethanol and diamino compounds such as ethylenediamine, 1,2-propylenediamine, 1,5-pentanediamine, 1,6-hexanediamine, and the like. The noscapine analogs may then be coupled to the linker by first forming the succinated derivative with succinic anhydride followed by condensation with the linker with DCC, EDC or EDCI.

In addition, the noscapine analogs may be oxidized with periodate and the resulting dialdehyde condensed with an amino alcohol or diamino compound listed above. The free hydroxyl or amino group on the linker may then be condensed with the succinate derivative of the antigen in the presence of DCC, EDC or EDCI. Many types of linkers are known in the art and may be used in the creation of conjugates. A non-limiting list of exemplary linkers is shown in Table I.

TABLE 1
Examples of hetero-bifunctional cross linking agents
Hetero-Bifunctional Cross Linking Agents

| | | | Spacer Arm |
|---|---|---|---|
| | | | Length after |
| | | Advantages and | crosslinking |
| Linker | Reactive Toward | Applications | (angstroms) |
| SMPT | Primary amines | Greater stability | 11.2 |
| | Sulfhydryls | | |
| SPDP | Primary amines | Thiolation | 6.8 |
| | Sulfhydryls | Cleavable cross- | |
| | | linking | |
| LC-SPDP | Primary amines | Extended spacer | 15.6 |
| | Sulfhydryls | arm | |
| Sulfo-LC-SPDP | Primary amines | Extended spacer | 15.6 |
| | Sulfhydryls | arm | |
| | | Water soluble | |
| SMCC | Primary amines | Stable maleimide | 11.6 |
| | Sulfhydryls | reactive group | |
| | | Enzyme- | |
| | | antibody | |
| | | conjugation | |
| | | Hapten-carrier | |
| | | protein | |
| | | conjugation | |
| Sulfo-SMCC | Primary amines | Stable maleimide | 11.6 |
| | Sulfhydryls | reactive group | |
| | | Enzyme- | |
| | | antibody | |
| | | conjugation | |
| MBS | Primary amines | Enzyme- | 9.9 |
| | Sulfhydryls | antibody | |
| | | conjugation | |
| | | Hapten-carrier | |
| | | protein | |
| | | conjugation | |
| Sulfo-MBS | Primary amines | Water soluble | 9.9 |
| | Sulfhydryls | | |
| SIAB | Primary amines | Enzyme- | 10.6 |
| | Sulfhydryls | antibody | |
| | | conjugation | |
| Sulfo-SIAB | Primary amines | Water soluble | 10.6 |
| | Sulfhydryls | | |
| SMPB | Primary amines | Extended spacer | 14.5 |
| | Sulfhydryls | arm | |
| | | Enzyme- | |
| | | antibody | |
| | | conjugation | |
| Sulfo-SMPB | Primary amines | Extended spacer | 14.5 |
| | Sulfhydryls | arm | |
| | | Water-soluble | |
| EDC/Sulfo-NHS | Primary amines | Hapten-carrier | 0 |
| | Carboxyl groups | conjugation | |
| ABH | Carbohydrates | Reacts with | 11.9 |
| | Non-selective | sugar moieties | |

### III. Pharmaceutical Compositions

The compounds described herein can be formulated for enteral, parenteral, topical, or pulmonary administration. The compounds can be combined with one or more pharmaceutically acceptable carriers and/or excipients that are considered safe and effective and may be administered to an individual without causing undesirable biological side effects or unwanted interactions. The carrier is all components present in the pharmaceutical formulation other than the active ingredient or ingredients.

A. Parenteral Formulations

The compounds described herein can be formulated for parenteral administration. “Parenteral administration”, as used herein, means administration by any method other than through the digestive tract or non-invasive topical or regional routes. For example, parenteral administration may include administration to a patient intravenously, intradermally, intraarterially, intraperitoneally, intralesionally, intracranially, intraarticularly, intraprostatically, intrapleurally, intratracheally, intravitreally, intratumorally, intramuscularly, subcutaneously, subconjunctivally, intravesicularly, intrapericardially, intraumbilically, by injection, and by infusion.

Parenteral formulations can be prepared as aqueous compositions using techniques is known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, viscosity modifying agents, and combination thereof.

Suitable surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sultanate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

The formulation is typically buffered to a pH of 3-8 for parenteral administration upon reconstitution. Suitable buffers include, but are not limited to, phosphate buffers, acetate buffers, and citrate buffers.

Water soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The powders can be prepared in such a manner that the particles are porous in nature, which can increase dissolution of the particles. Methods for making porous particles are well known in the art.

1. Controlled Release Formulations

The parenteral formulations described herein can be formulated for controlled release including immediate release, delayed release, extended release, pulsatile release, and combinations thereof.

i. Nano- and Microparticles

For parenteral administration, the one or more noscapine analogs, and optional one or more additional active agents, can be incorporated into microparticles, nanoparticles, or combinations thereof that provide controlled release of the noscapine analogs and/or one or more additional active agents. In embodiments wherein the formulations contains two or more drugs, the drugs can be formulated for the same type of controlled release (e.g., delayed, extended, immediate, or pulsatile) or the drugs can be independently formulated for different types of release (e.g., immediate and delayed, immediate and extended, delayed and extended, delayed and pulsatile, etc.).

For example, the noscapine analogs and/or one or more additional active agents can be incorporated into polymeric microparticles which provide controlled release of the drug(s). Release of the drug(s) is controlled by diffusion of the drug(s) out of the microparticles and/or degradation of the polymeric particles by hydrolysis and/or enzymatic degradation. Suitable polymers include ethylcellulose and other natural or synthetic cellulose derivatives.

Polymers which are slowly soluble and form a gel in an aqueous environment, such as hydroxypropyl methylcellulose or polyethylene oxide may also be suitable as materials for drug containing microparticles. Other polymers include, but are not limited to, polyanhydrides, polyester anhydrides), polyhydroxy acids, such as polylactide (PLA), polyglycolide (PGA), poly(lactide-co-glycolide) (PLGA), poly-3-hydroxybutyrate (PHB) and copolymers thereof, poly-4-hydroxybutyrate (P4HB) and copolymers thereof, polycaprolactone and copolymers thereof, and combinations thereof.

Alternatively, the drug(s) can be incorporated into microparticles prepared from materials which are insoluble in aqueous solution or slowly soluble in aqueous solution, but are capable of degrading within the GI tract by means including enzymatic degradation, surfactant action of bile acids, and/or mechanical erosion. As used herein, the term “slowly soluble in water” refers to materials that are not dissolved in water within a period of 30 minutes. Preferred examples include fats, fatty substances, waxes, wax-like substances and mixtures thereof. Suitable fats and fatty substances include fatty alcohols (such as lauryl, myristyl stearyl, cetyl or cetostearyl alcohol), fatty acids and derivatives, including but not limited to fatty acid esters, fatty acid glycerides (mono-, di- and tri-glycerides), and hydrogenated fats. Specific examples include, but are not limited to hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes. Specific examples of waxes include beeswax, glycowax, castor wax, carnauba wax, paraffins and candelilla wax. As used herein, a wax-like material is defined as any material which is normally solid at room temperature and has a melting point of from about 30 to 300° C.

In some cases, it may be desirable to alter the rate of water penetration into the microparticles. To this end, rate-controlling (wicking) agents may be formulated along with the fats or waxes listed above. Examples of rate-controlling materials include certain starch derivatives (e.g., waxy maltodextrin and drum dried corn starch), cellulose derivatives (e.g., hydroxypropylmethyl-cellulose, hydroxypropylcellulose, methylcellulose, and carboxymethyl-cellulose), alginic acid, lactose and talc. Additionally, a pharmaceutically acceptable surfactant (for example, lecithin) may be added to facilitate the degradation of such microparticles.

Proteins which are water insoluble, such as zein, can also be used as materials for the formation of drug containing microparticles. Additionally, proteins, polysaccharides and combinations thereof which are water soluble can be formulated with drug into microparticles and subsequently cross-linked to form an insoluble network. For example, cyclodextrins can be complexed with individual drug molecules and subsequently cross-linked.

Encapsulation or incorporation of drug into carrier materials to produce drug containing microparticles can be achieved through known pharmaceutical formulation techniques. In the case of formulation in fats, waxes or wax-like materials, the carrier material is typically heated above its melting temperature and the drug is added to form a mixture comprising drug particles suspended in the carrier material, drug dissolved in the carrier material, or a mixture thereof. Microparticles can be subsequently formulated through several methods including, but not limited to, the processes of congealing, extrusion, spray chilling or aqueous dispersion. In a preferred process, wax is heated above its melting temperature, drug is added, and the molten wax-drug mixture is congealed under constant stirring as the mixture cools. Alternatively, the molten wax-drug mixture can be extruded and spheronized to form pellets or beads. These processes are known in the art.

For some carrier materials it may be desirable to use a solvent evaporation technique to produce drug containing microparticles. In this case drug and carrier material are co-dissolved in a mutual solvent and microparticles can subsequently be produced by several techniques including, but not limited to, forming an emulsion in water or other appropriate media, spray drying or by evaporating off the solvent from the bulk solution and milling the resulting material.

In some embodiments, drug in a particulate form is homogeneously dispersed in a water-insoluble or slowly water soluble material. To minimize the size of the drug particles within the composition, the drug powder itself may be milled to generate fine particles prior to formulation. The process of jet milling, known in the pharmaceutical art, can be used for this purpose. In some embodiments drug in a particulate form is homogeneously dispersed in a wax or wax like substance by heating the wax or wax like substance above its melting point and adding the drug particles while stirring the mixture. In this case a pharmaceutically acceptable surfactant may be added to the mixture to facilitate the dispersion of the drug particles.

The particles can also be coated with one or more modified release coatings. Solid esters of fatty acids, which are hydrolyzed by lipases, can be spray coated onto microparticles or drug particles. Zein is an example of a naturally water-insoluble protein. It can be coated onto drug containing microparticles or drug particles by spray coating or by wet granulation techniques. In addition to naturally water-insoluble materials, some substrates of digestive enzymes can be treated with cross-linking procedures, resulting in the formation of non-soluble networks. Many methods of cross-linking proteins, initiated by both chemical and physical means, have been reported. One of the most common methods to obtain cross-linking is the use of chemical cross-linking agents. Examples of chemical cross-linking agents include aldehydes (gluteraldehyde and formaldehyde), epoxy compounds, carbodiimides, and genipin. In addition to these cross-linking agents, oxidized and native sugars have been used to cross-link gelatin. Cross-linking can also be accomplished using enzymatic means; for example, transglutaminase has been approved as a GRAS substance for cross-linking seafood products. Finally, cross-linking can be initiated by physical means such as thermal treatment, UV irradiation and gamma irradiation.

To produce a coating layer of cross-linked protein surrounding drug containing microparticles or drug particles, a water soluble protein can be spray coated onto the microparticles and subsequently cross-linked by the one of the methods described above. Alternatively, drug containing microparticles can be microencapsulated within protein by coacervation-phase separation (for example, by the addition of salts) and subsequently cross-linked. Some suitable proteins for this purpose include gelatin, albumin, casein, and gluten.

Polysaccharides can also be cross-linked to form a water-insoluble network. For many polysaccharides, this can be accomplished by reaction with calcium salts or multivalent cations which cross-link the main polymer chains. Pectin, alginate, dextran, amylose and guar gum are subject to cross-linking in the presence of multivalent cations. Complexes between oppositely charged polysaccharides can also be formed; pectin and chitosan, for example, can be complexed via electrostatic interactions.

In certain embodiments, it may be desirable to provide continuous delivery of one or more noscapine analogs to a patient in need thereof. For intravenous or intraarterial routes, this can be accomplished using drip systems, such as by intravenous administration. For topical applications, repeated application can be done or a patch can be used to provide continuous administration of the noscapine analogs over an extended period of time.

2. Injectable/Implantable Solid Implants

The noscapine analogs described herein can be incorporated into injectable/implantable solid or semi-solid implants, such as polymeric implants. In one embodiment, the noscapine analogs are incorporated into a polymer that is a liquid or paste at room temperature, but upon contact with aqueous medium, such as physiological fluids, exhibits an increase in viscosity to form a semi-solid or solid material. Exemplary polymers include, but are not limited to, hydroxyalkanoic acid polyesters derived from the copolymerization of at least one unsaturated hydroxy fatty acid copolymerized with hydroxyalkanoic acids. The polymer can be melted, mixed with the active substance and cast or injection molded into a device. Such melt fabrication require polymers having a melting point that is below the temperature at which the substance to be delivered and polymer degrade or become reactive. The device can also be prepared by solvent casting where the polymer is dissolved in a solvent and the drug dissolved or dispersed in the polymer solution and the solvent is then evaporated. Solvent processes require that the polymer be soluble in organic solvents. Another method is compression molding of a mixed powder of the polymer and the drug or polymer particles loaded with the active agent.

Alternatively, the noscapine analogs can be incorporated into a polymer matrix and molded, compressed, or extruded into a device that is a solid at room temperature. For example, the noscapine analogs can be incorporated into a biodegradable polymer, such as polyanhydrides, polyhydroalkanoic acids (PHAs), PLA, PGA, PLGA, polycaprolactone, polyesters, polyamides, polyorthoesters, polyphosphazenes, proteins and polysaccharides such as collagen, hyaluronic acid, albumin and gelatin, and combinations thereof and compressed into solid device, such as disks, or extruded into a device, such as rods.

The release of the one or more noscapine analogs from the implant can be varied by selection of the polymer, the molecular weight of the polymer, and/or modification of the polymer to increase degradation, such as the formation of pores and/or incorporation of hydrolyzable linkages. Methods for modifying the properties of biodegradable polymers to vary the release profile of the noscapine analogs from the implant are well known in the art.

B. Enteral Formulations

Suitable oral dosage fauns include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art.

Formulations may be prepared using a pharmaceutically acceptable carrier. As generally used herein “carrier” includes, but is not limited to, diluents, preservatives, binders, lubricants, disintegrators, swelling agents, fillers, stabilizers, and combinations thereof.

Carrier also includes all components of the coating composition which may include plasticizers, pigments, colorants, stabilizing agents, and glidants. Delayed release dosage formulations may be prepared as described in standard references. These references provide information on carriers, materials, equipment and process for preparing tablets and capsules and delayed release dosage forms of tablets, capsules, and granules.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

Optional pharmaceutically acceptable excipients include, but are not limited to, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone® XL from GAF Chemical Corp).

Stabilizers are used to inhibit or retard drug decomposition reactions which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

i. Controlled Release Formulations

Oral dosage forms, such as capsules, tablets, solutions, and suspensions, can for formulated for controlled release. For example, the one or more noscapine analogs and optional one or more additional active agents can be formulated into nanoparticles, microparticles, and combinations thereof, and encapsulated in a soft or hard gelatin or non-gelatin capsule or dispersed in a dispersing medium to form an oral suspension or syrup. The particles can be formed of the drug and a controlled release polymer or matrix. Alternatively, the drug particles can be coated with one or more controlled release coatings prior to incorporation in to the finished dosage form.

In another embodiment, the one or more noscapine analogs and optional one or more additional active agents are dispersed in a matrix material, which gels or emulsifies upon contact with an aqueous medium, such as physiological fluids. In the case of gels, the matrix swells entrapping the active agents, which are released slowly over time by diffusion and/or degradation of the matrix material. Such matrices can be formulated as tablets or as fill materials for hard and soft capsules.

In still another embodiment, the one or more noscapine analogs, and optional one or more additional active agents are formulated into a sold oral dosage form, such as a tablet or capsule, and the solid dosage form is coated with one or more controlled release coatings, such as a delayed release coatings or extended release coatings. The coating or coatings may also contain the noscapine analogs and/or additional active agents.

Extended Release Dosage Forms

The extended release formulations are generally prepared as diffusion or osmotic systems, which are known in the art. A diffusion system typically consists of two types of devices, a reservoir and a matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, cellulosic polymers such as methyl and ethyl cellulose, hydroxyalkylcelluloses such as hydroxypropyl-cellulose, hydroxypropylmethylcellulose, sodium carboxymethylcellulose, and Carbopol® 934, polyethylene oxides and mixtures thereof. Fatty compounds include, but are not limited to, various waxes such as carnauba wax and glyceryl tristearate and wax-type substances including hydrogenated castor oil or hydrogenated vegetable oil, or mixtures thereof.

In certain preferred embodiments, the plastic material is a pharmaceutically acceptable acrylic polymer, including but not limited to, acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer poly(methyl methacrylate), poly(methacrylic acid) (anhydride), polymethacrylate, polyacrylamide, poly(methacrylic acid anhydride), and glycidyl methacrylate copolymers.

In certain preferred embodiments, the acrylic polymer is comprised of one or more ammonia methacrylate copolymers. Ammonia methacrylate copolymers are well known in the art, and are described in NF XVII as fully polymerized copolymers of acrylic and methacrylic acid esters with a low content of quaternary ammonium groups.

In one preferred embodiment, the acrylic polymer is an acrylic resin lacquer such as that which is commercially available from Rohm Pharma under the tradename Eudragit®. In further preferred embodiments, the acrylic polymer comprises a mixture of two acrylic resin lacquers commercially available from Rohm Pharma under the tradenames Eudragit® RL30D and Eudragit® RS30D, respectively. Eudragit® RL30D and Eudragit® RS30D are copolymers of acrylic and methacrylic esters with a low content of quaternary ammonium groups, the molar ratio of ammonium groups to the remaining neutral (meth)acrylic esters being 1:20 in Eudragit® RL30D and 1:40 in Eudragit® RS30D. The mean molecular weight is about 150,000. Eudragit® S-100 and Eudragit® L-100 are also preferred. The code designations RL (high permeability) and RS (low permeability) refer to the permeability properties of these agents. Eudragit® RL/RS mixtures are insoluble in water and in digestive fluids. However, multiparticulate systems formed to include the same are swellable and permeable in aqueous solutions and digestive fluids.

The polymers described above such as Eudragit® RL/RS may be mixed together in any desired ratio in order to ultimately obtain a sustained-release formulation having a desirable dissolution profile. Desirable sustained-release multiparticulate systems may be obtained, for instance, from 100% Eudragit® RL, 50% Eudragit® RL and 50% Eudragit® RS, and 10% Eudragit® RL and 90% Eudragit® RS. One skilled in the art will recognize that other acrylic polymers may also be used, such as, for example, Eudragit® L.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above can be combined in a final dosage form comprising single or multiple units. Examples of multiple units include, but are not limited to, multilayer tablets and capsules containing tablets, beads, or granules. An immediate release portion can be added to the extended release system by means of either applying an immediate release layer on top of the extended release core using a coating or compression process or in a multiple unit system such as a capsule containing extended and immediate release beads.

Extended release tablets containing hydrophilic polymers are prepared by techniques commonly known in the art such as direct compression, wet granulation, or dry granulation. Their formulations usually incorporate polymers, diluents, binders, and lubricants as well as the active pharmaceutical ingredient. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In the congealing method, the drug is mixed with a wax material and either spray-congealed or congealed and screened and processed.

Delayed Release Dosage Forms

Delayed release formulations can be created by coating a solid dosage form with a polymer film, which is insoluble in the acidic environment of the stomach, and soluble in the neutral environment of the small intestine.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit® (Rohm Pharma; Westerstadt, Germany), including Eudragit® L30D-55 and L100-55 (soluble at pH 5.5 and above), Eudragit® L-100 (soluble at pH 6.0 and above), Eudragit® S (soluble at pH 7.0 and above, as a result of a higher degree of esterification), and Eudragits® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

C. Topical Formulations

Suitable dosage forms for topical administration include creams, ointments, salves, sprays, gels, lotions, emulsions, and transdermal patches.

The formulation may be formulated for transmucosal, transepithelial, transendothelial, or transdermal administration. The compounds can also be formulated for intranasal delivery, pulmonary delivery, or inhalation. The compositions may further contain one or more chemical penetration enhancers, membrane permeability agents, membrane transport agents, emollients, surfactants, stabilizers, and combination thereof.

1. Topical Formulations “Emollients” are an externally applied agent that softens or soothes skin and are generally known in the art and listed in compendia, such as the “Handbook of Pharmaceutical Excipients”, 4^{th} Ed., Pharmaceutical Press, 2003. These include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In one embodiment, the emollients are ethylhexylstearate and ethylhexyl palmitate.

“Surfactants” are surface-active agents that lower surface tension and thereby increase the emulsifying, foaming, dispersing, spreading and wetting properties of a product. Suitable non-ionic surfactants include emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In one embodiment, the non-ionic surfactant is stearyl alcohol.

“Emulsifiers” are surface active substances which promote the suspension of one liquid in another and promote the formation of a stable mixture, or emulsion, of oil and water. Common emulsifiers are: metallic soaps, certain animal and vegetable oils, and various polar compounds. Suitable emulsifiers include acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In one embodiment, the emulsifier is glycerol stearate.

Suitable classes of penetration enhancers are known in the art and include, but are not limited to, fatty alcohols, fatty acid esters, fatty acids, fatty alcohol ethers, amino acids, phospholipids, lecithins, cholate salts, enzymes, amines and amides, complexing agents (liposomes, cyclodextrins, modified celluloses, and diimides), macrocyclics, such as macrocyclic lactones, ketones, and anhydrides and cyclic ureas, surfactants, N-methyl pyrrolidones and derivatives thereof, DMSO and related compounds, ionic compounds, azone and related compounds, and solvents, such as alcohols, ketones, amides, polyols (e.g., glycols). Examples of these classes are known in the art.

i. Lotions, Creams, Gels, Ointments, Emulsions, and Foams

“Hydrophilic” as used herein refers to substances that have strongly polar groups that readily interact with water.

“Lipophilic” refers to compounds having an affinity for lipids.

“Amphiphilic” refers to a molecule combining hydrophilic and lipophilic (hydrophobic) properties

“Hydrophobic” as used herein refers to substances that lack an affinity for water; tending to repel and not absorb water as well as not dissolve in or mix with water.

A “gel” is a colloid in which the dispersed phase has combined with the continuous phase to produce a semisolid material, such as jelly.

An “oil” is a composition containing at least 95% wt of a lipophilic substance. Examples of lipophilic substances include but are not limited to naturally occurring and synthetic oils, fats, fatty acids, lecithins, triglycerides and combinations thereof.

A “continuous phase” refers to the liquid in which solids are suspended or droplets of another liquid are dispersed, and is sometimes called the external phase. This also refers to the fluid phase of a colloid within which solid or fluid particles are distributed. If the continuous phase is water (or another hydrophilic solvent), water-soluble or hydrophilic drugs will dissolve in the continuous phase (as opposed to being dispersed). In a multiphase formulation (e.g., an emulsion), the discreet phase is suspended or dispersed in the continuous phase.

An “emulsion” is a composition containing a mixture of non-miscible components homogenously blended together. In particular embodiments, the non-miscible components include a lipophilic component and an aqueous component. An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. The oil phase may consist at least in part of a propellant, such as an HFA propellant. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

A sub-set of emulsions are the self-emulsifying systems. These drug delivery systems are typically capsules (hard shell or soft shell) comprised of the drug dispersed or dissolved in a mixture of surfactant(s) and lipophilic liquids such as oils or other water immiscible liquids. When the capsule is exposed to an aqueous environment and the outer gelatin shell dissolves, contact between the aqueous medium and the capsule contents instantly generates very small emulsion droplets. These typically are in the size range of micelles or nanoparticles. No mixing force is required to generate the emulsion as is typically the case in emulsion formulation processes.

A “lotion” is a low- to medium-viscosity liquid formulation. A lotion can contain finely powdered substances that are in soluble in the dispersion medium through the use of suspending agents and dispersing agents. Alternatively, lotions can have as the dispersed phase liquid substances that are immiscible with the vehicle and are usually dispersed by means of emulsifying agents or other suitable stabilizers. In one embodiment, the lotion is in the form of an emulsion having a viscosity of between 100 and 1000 centistokes. The fluidity of lotions permits rapid and uniform application over a wide surface area. Lotions are typically intended to dry on the skin leaving a thin coat of their medicinal components on the skin's surface.

A “cream” is a viscous liquid or semi-solid emulsion of either the “oil-in-water” or “water-in-oil type”. Creams may contain emulsifying agents and/or other stabilizing agents. In one embodiment, the formulation is in the form of a cream having a viscosity of greater than 1000 centistokes, typically in the range of 20,000-50,000 centistokes. Creams are often time preferred over ointments as they are generally easier to spread and easier to remove.

The difference between a cream and a lotion is the viscosity, which is dependent on the amount/use of various oils and the percentage of water used to prepare the formulations. Creams are typically thicker than lotions, may have various uses and often one uses more varied oils/butters, depending upon the desired effect upon the skin. In a cream formulation, the water-base percentage is about 60-75% and the oil-base is about 20-30% of the total, with the other percentages being the emulsifier agent, preservatives and additives for a total of 100%.

An “ointment” is a semisolid preparation containing an ointment base and optionally one or more active agents. Examples of suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy that ointments prepared with the same components.

A “gel” is a semisolid system containing dispersions of small or large molecules in a liquid vehicle that is rendered semisolid by the action of a thickening agent or polymeric material dissolved or suspended in the liquid vehicle. The liquid may include a lipophilic component, an aqueous component or both. Some emulsions may be gels or otherwise include a gel component. Some gels, however, are not emulsions because they do not contain a homogenized blend of immiscible components. Suitable gelling agents include, but are not limited to, modified celluloses, such as hydroxypropyl cellulose and hydroxyethyl cellulose; Carbopol homopolymers and copolymers; and combinations thereof. Suitable solvents in the liquid vehicle include, but are not limited to, diglycol monoethyl ether; alklene glycols, such as propylene glycol; dimethyl isosorbide; alcohols, such as isopropyl alcohol and ethanol. The solvents are typically selected for their ability to dissolve the chug. Other additives, which improve the skin feel and/or emolliency of the formulation, may also be incorporated. Examples of such additives include, but are not limited, isopropyl myristate, ethyl acetate, C₁₂-C₁₅ alkyl benzoates, mineral oil, squalane, cyclomethicone, capric/caprylic triglycerides, and combinations thereof.

Foams consist of an emulsion in combination with a gaseous propellant. The gaseous propellant consists primarily of hydrofluoroalkanes (HFAs). Suitable propellants include HFAs such as 1,1,1,2-tetrafluoroethane (HFA 134a) and 1,1,1,2,3,3,3-heptafluoropropane (HFA 227), but mixtures and admixtures of these and other HFAs that are currently approved or may become approved for medical use are suitable. The propellants preferably are not hydrocarbon propellant gases which can produce flammable or explosive vapors during spraying. Furthermore, the compositions preferably contain no volatile alcohols, which can produce flammable or explosive vapors during use.

Buffers are used to control pH of a composition. Preferably, the buffers buffer the composition from a pH of about 4 to a pH of about 7.5, more preferably from a pH of about 4 to a pH of about 7, and most preferably from a pH of about 5 to a pH of about 7. In a preferred embodiment, the buffer is triethanolamine.

Preservatives can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal.

In certain embodiments, it may be desirable to provide continuous delivery of one or more noscapine analogs to a patient in need thereof. For topical applications, repeated application can be done or a patch can be used to provide continuous administration of the noscapine analogs over an extended period of time.

D. Pulmonary Formulations

In one embodiment, the noscapine analogs are formulated for pulmonary delivery, such as intranasal administration or oral inhalation. The respiratory tract is the structure involved in the exchange of gases between the atmosphere and the blood stream. The lungs are branching structures ultimately ending with the alveoli where the exchange of gases occurs. The alveolar surface area is the largest in the respiratory system and is where drug absorption occurs. The alveoli are covered by a thin epithelium without cilia or a mucus blanket and secrete surfactant phospholipids.

The respiratory tract encompasses the upper airways, including the oropharynx and larynx, followed by the lower airways, which include the trachea followed by bifurcations into the bronchi and bronchioli. The upper and lower airways are called the conducting airways. The terminal bronchioli then divide into respiratory bronchioli which then lead to the ultimate respiratory zone, the alveoli, or deep lung. The deep lung, or alveoli, are the primary target of inhaled therapeutic aerosols for systemic drug delivery.

Pulmonary administration of therapeutic compositions comprised of low molecular weight drugs has been observed, for example, beta-androgenic antagonists to treat asthma. Other therapeutic agents that are active in the lungs have been administered systemically and targeted via pulmonary absorption. Nasal delivery is considered to be a promising technique for administration of therapeutics for the following reasons: the nose has a large surface area available for drug absorption due to the coverage of the epithelial surface by numerous microvilli, the subepithelial layer is highly vascularized, the venous blood from the nose passes directly into the systemic circulation and therefore avoids the loss of drug by first-pass metabolism in the liver, it offers lower doses, more rapid attainment of therapeutic blood levels, quicker onset of pharmacological activity, fewer side effects, high total blood flow per cm³, porous endothelial basement membrane, and it is easily accessible.

The term aerosol as used herein refers to any preparation of a fine mist of particles, which can be in solution or a suspension, whether or not it is produced using a propellant. Aerosols can be produced using standard techniques, such as ultrasonication or high pressure treatment.

Carriers for pulmonary formulations can be divided into those for dry powder formulations and for administration as solutions. Aerosols for the delivery of therapeutic agents to the respiratory tract are known in the art. For administration via the upper respiratory tract, the formulation can be formulated into a solution, e.g., water or isotonic saline, buffered or unbuffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. For example, a representative nasal decongestant is described as being buffered to a pH of about 6.2. One skilled in the art can readily determine a suitable saline content and pH for an innocuous aqueous solution for nasal and/or upper respiratory administration.

Preferably, the aqueous solutions is water, physiologically acceptable aqueous solutions containing salts and/or buffers, such as phosphate buffered saline (PBS), or any other aqueous solution acceptable for administration to a animal or human. Such solutions are well known to a person skilled in the art and include, but are not limited to, distilled water, de-ionized water, pure or ultrapure water, saline, phosphate-buffered saline (PBS). Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

In another embodiment, solvents that are low toxicity organic (i.e. nonaqueous) class 3 residual solvents, such as ethanol, acetone, ethyl acetate, tetrahydrofuran, ethyl ether, and propanol may be used for the formulations. The solvent is selected based on its ability to readily aerosolize the formulation. The solvent should not detrimentally react with the noscapine analogs. An appropriate solvent should be used that dissolves the noscapine analogs or forms a suspension of the noscapine analogs. The solvent should be sufficiently volatile to enable formation of an aerosol of the solution or suspension. Additional solvents or aerosolizing agents, such as freons, can be added as desired to increase the volatility of the solution or suspension.

In one embodiment, compositions may contain minor amounts of polymers, surfactants, or other excipients well known to those of the art. In this context, “minor amounts” means no excipients are present that might affect or mediate uptake of the noscapine analogs in the lungs and that the excipients that are present are present in amount that do not adversely affect uptake of noscapine analogs in the lungs.

Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial. The film swells easily when reconstituted with ethanol. To fully disperse the lipid molecules in the organic solvent, the suspension is sonicated. Nonaqueous suspensions of lipids can also be prepared in absolute ethanol using a reusable PART LC Jet+ nebulizer (PARI Respiratory Equipment, Monterey, Calif.).

Dry powder formulations (“DPFs”) with large particle size have improved flowability characteristics, such as less aggregation, easier aerosolization, and potentially less phagocytosis. Dry powder aerosols for inhalation therapy are generally produced with mean diameters primarily in the range of less than 5 microns, although a preferred range is between one and ten microns in aerodynamic diameter. Large “carrier” particles (containing no drug) have been co-delivered with therapeutic aerosols to aid in achieving efficient aerosolization among other possible benefits.

Polymeric particles may be prepared using single and double emulsion solvent evaporation, spray drying, solvent extraction, solvent evaporation, phase separation, simple and complex coacervation, interfacial polymerization, and other methods well known to those of ordinary skill in the art. Particles may be made using methods for making microspheres or microcapsules known in the art. The preferred methods of manufacture are by spray drying and freeze drying, which entails using a solution containing the surfactant, spraying to form droplets of the desired size, and removing the solvent.

The particles may be fabricated with the appropriate material, surface roughness, diameter and tap density for localized delivery to selected regions of the respiratory tract such as the deep lung or upper airways. For example, higher density or larger particles may be used for upper airway delivery. Similarly, a mixture of different sized particles, provided with the same or different EGS may be administered to target different regions of the lung in one administration.

Formulations for pulmonary delivery include unilamellar phospholipid vesicles, liposomes, or lipoprotein particles. Formulations and methods of making such formulations containing nucleic acid are well known to one of ordinary skill in the art. Liposomes are formed from commercially available phospholipids supplied by a variety of vendors including Avanti Polar Lipids, Inc. (Birmingham, Ala.). In one embodiment, the liposome can include a ligand molecule specific for a receptor on the surface of the target cell to direct the liposome to the target cell.

E. Other Active Agents

The noscapine analogs described herein can be co-administered with one or more additional active agents, such as diagnostic agents, therapeutic agents, and/or prophylactic agents. Suitable classes of active agents include, but are not limited to:

Alkylating agents, such as nitrogen mustards (e.g., mechloroethamine, cyclophosphamide, ifosfamide, melphalan, and chlorambucil), ethylenimines and methylmelamines (e.g., heaxamethylmelamine), alkyl sulfonates (e.g., thiotepa and busulfan) nitrosoureas (e.g., carmustine, lomustine, semustine, and streptozocin), and triazines (e.g., dacarbazine);

Antimetabolites, such as folic acid and analogs thereof (e.g., methotrexate), pyrimidine analogs (e.g., fluoracil, floxuridine, and cytarabine), purine analogs and related inhibitors (e.g., mercaptopurine, thioguanine, and pentostatin),
Cytotoxic anticancer agents, such as paclitaxel;
Cytostatic and/or cytotoxic agents such as anti-angiogenic agents such as endostatin, angiostatin, thalidomide;
Analgesics, such as opioid and non-opioid analgesics; and
Vaccines containing cancer antigens or immunomodulators such as cytokines to enhance the anti-cancer activity;

Natural products, such as vinca alkaloids (e.g., vinblastine and vincristine), epipodophyllotoxins (e.g., etoposide and tertiposide), antibiotics (e.g., dactinomycin, daunorubicin, doxorubicin, bleomycin, plicamycin, and mitomycin), enzymes (e.g., L-asparaginase), and biological response modifiers (e.g., interferon alpha);

Proteasome inhibitors, such as lactacystin, MG-132, and PS-341;

Tyrosine kinase inhibitors, such as Gleevec®, ZD 1839 (Iressa®), SH268, genistein, CEP2563, SU6668, SU1 1248, and EMD121974;

Retinoids and synthetic retinoids, such as bexarotene, tretinoin, 13-cis-retinoic acid, 9-cis-retinoic acid, .alpha.-difluoromethylornithine, ILX23-7553, fenretinide, and N-4-carboxyphenyl retinamide;

Cyclin-dependent kinase inhibitors, such as flavopiridol, UCN-01, roscovitine and olomoucine;

COX-2 inhibitors include, such as celecoxib, valecoxib, and rofecoxib;

Prenylprotein transferase inhibitors, such as R1 15777, SCH66336, L-778,123, BAL9611 and TAN-1813;

Hormones and antagonists, such as adrenocorticosteroids (e.g., prednisone), progestins (e.g, hydroxyprogesterone caproate, medroxyprogesterone acetate, and megestrol acetate), estrogens (e.g., diethylstilbestrol and ethinyl estradiol), antiestrogen (e.g., tamoxifen), androgens (e.g., testosterone propionate, fluoxtnesterone, antiandrogen), and gonadotropin-releasing hormone analogs;

Sigma-2 receptor agonists, such as CB-64D, CB-184 and haloperidol;

HMG-CoA reductase inhibitors, such as lovastatin, simvastatin, pravastatin, fluvastatin, atorvastatin and cerivastatin;

HIV protease inhibitors, such as amprenavir, abacavir, CGP-73547, CGP-61755, DMP-450, indinavir, nelfinavir, tipranavir, ritonavir, saquinavir, ABT-378, AG 1776, and BMS-232,632;

Proteins, such as insulin, and

Miscellaneous compounds, such as platinum coordination complexes (e.g., cisplatin and carboplatin), anthracenedione (e.g., mitoxantrone), substituted urea (hydroxyurea), methyl hydrazines (e.g., procarbazine), and adrenocortical suppressants (e.g., mitotane and aminogluethimide).

The one or more noscapine analogs and the one or more additional active agents can be formulated in the same dosage form or separate dosage forms. Alternatively, the one or more additional active agents can be administered simultaneously or almost simultaneously in different dosage forms. If in separate dosage units, the one or more noscapine analogs and the one or more additional active agents can be administered by the same route of administration or by different routes of administration. For example, the one or more noscapine analogs and the one or more additional active agents can both be administered parenterally, or one can be administered parenterally and one orally.

If the one or more noscapine analogs and the one or more active agents are administered sequentially, the second agent to be administered is administered typically less than 6 hours following administration of the first agent, preferably less than 4 hours after the first agent, more preferably less than 2 hours after the first agent, more preferably less than 1 hour after the first agent, most preferably less than 30 minutes after administration of the first agent, and most preferably immediately after administration of the first agent. “Immediately”, as used here, means less than 10 minutes, preferably less than 5 minutes, more preferably less than 2 minutes, most preferably less than one minute.

The noscapine analogs and the one or more additional active agents can be formulated for controlled release, for example, immediate release, delayed release, extended release, pulsatile release, and combinations thereof. In one embodiment, the one or more noscapine analogs are formulated for immediate release and the one or more additional agents are formulated for delayed, extended, or pulsatile release. In another embodiment, the one or more noscapine analogs are formulated for delayed, extended, or pulsatile release and the one or more additional active agents are formulated for immediate release. In still another embodiment, the one or more noscapine analogs and the one or more additional active agents are independently formulated for delayed, extended, or pulsatile release.

### IV. Methods of Making the Compounds

The synthetic scheme for preparing the benzofuranone analogs of Noscapine described herein is shown below.

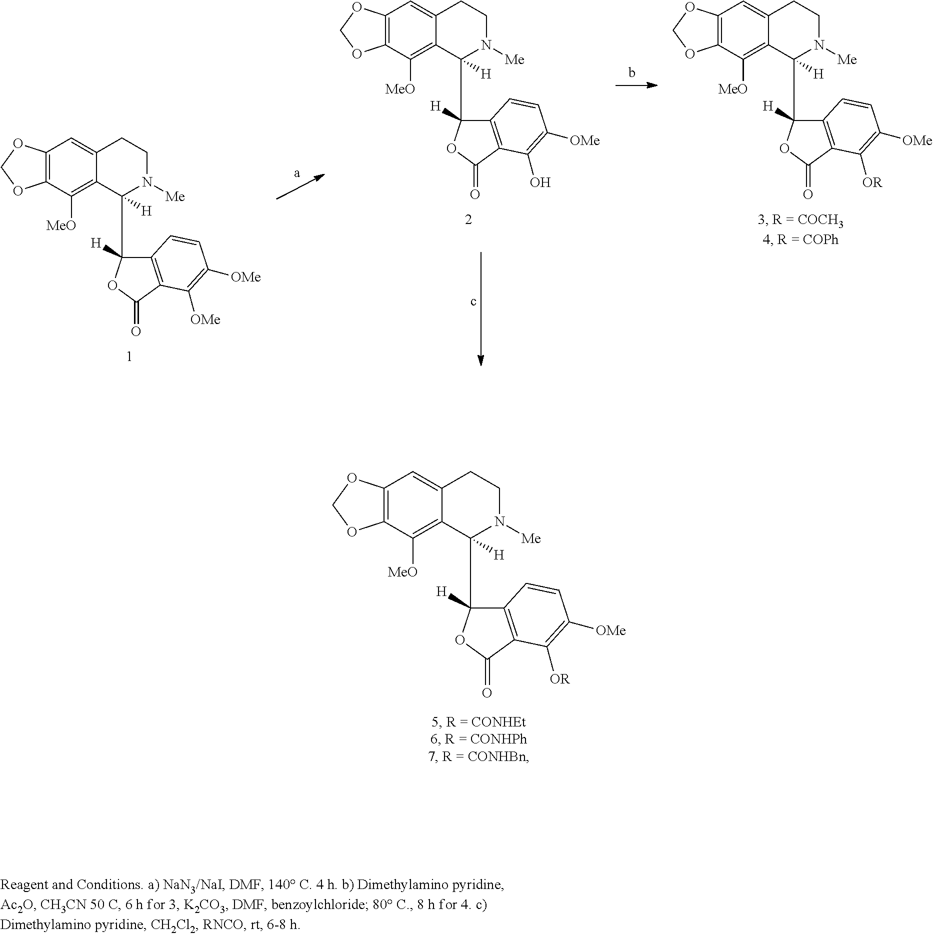

Sodium azide and sodium iodide in dimethylformamide (DMF) was selectively used to cleave the methyl group at position-7 of benzofuranone ring. As a result, an efficient method to prepare compound 2 (7-hydroxy noscapine) was developed that excluded the use of Grignard reagent and simplified the work-up procedure to obtain the reaction product. Briefly, noscapine was dissolved in anhydrous DMF along with sodium azide and sodium iodide and the mixture was stirred at 140° C. for 4 h. The mixture was condensed under reduced pressure and the residue was extracted in ethylacetate followed by washing with water and brine to remove excess salt. This synthetic method, offered a simple, economic and easy work-up procedure compared to the one reported in the literature. The 7-hydroxy noscapine, 2 thus obtained, served as a scaffold to synthesize various C-7-modified analogs of noscapine.

Two strategies were followed for the synthesis of 7-substituted noscapine analogs as depicted in Scheme 1. Starting from the key intermediate 2, in the first strategy, we performed acylation reactions using acetic anhydride and benzoyl chloride in the presence of a base to prepare compounds 3 and 4. Compound 3 is the 7-acetyl analog, which, in contrast to the almost inert original methoxy analog, has more polarized carbonyl functionality. Compound 4, a benzoyl analog, was prepared to compare the effect of alkyl to aryl function in the same molecule.

Carbamate esters can be used to mask free phenolic groups in biologically active compounds, such as anti-cancer agents. Thus, in the second strategy, a series of carbamate esters of the key intermediate, 2, were synthesized using readily available ethyl, phenyl and benzyl isocyanates. These compounds were prepared by the reaction of phenol analog 2 with various isocyanates in the presence of DMAP (4-N,N′-dimethylamino pyridine) in anhydrous dichloromethane. The partial hydrolysis of isocyanates led to the formation of urea impurities thus increasing the complexity of the purification process. Purification was accomplished by using repetitive flash chromatography.

### V. Methods of Using the Compounds

Noscapine and its analogs, collectively referred to as the noscapinoid family, typify a class of microtubule-modulating agents that evade the ‘harsher’ side-effects of currently-available tubulin-binding chemotherapeutics by preserving the total polymer mass of tubulin. This class of non-toxic microtubule-modulating agents is based upon the parent molecule, noscapine, a relatively innocuous, non-sedative, isoquinoline alkaloid from opium, known for its antitussive properties for decades. Unlike the two major classes of tubulin-binding drugs, which either overpolymerize and bundle microtubules (taxanes) or depolymerize them and form paracrystals (vincas), noscapine and its analogs do not exert gross affects on the microtubular ultrastructure. Thus, noscapine and its analogs generally do not impair crucial microtubule functions and cause minimal toxicity, if any, and are best characterized as ‘kinder and gentler’ microtubule-modulating agents. Since for clinical significance, the therapeutic efficacy is based upon the potency and selectivity (non-toxicity to normal cells), noscapine analogs can potentially be exploited for therapeutic usage individually or in combination with existing toxic anti-microtubule drugs.

In silica molecular modeling efforts predicted the rational design of novel second generation noscapine analogs substituted at position-7 of the benzofuranone ring system. Contrary to the hypothesis that increasing the steric bulk of the substituent at position 7 would negatively affect the activity of the compounds, several of the compounds were more effective than noscapine against certain cancer cell lines. Although each synthesized analog showed cytotoxicity activity within a narrow range for most cell lines, significant inter cell line variations were found to exist, in that a particular compound exhibited differential sensitivity in cell lines from varying tissue origin. These differences may be attributable to the presence of varying tubulin isotype expression and mutations in the tubulin gene in different cell types. It is also likely that altered expression of survival and drug resistance mechanisms in cell lines from different tissue types dictate cellular sensitivities.

The effects of various concentrations of five noscapine analogs on the polymerization of tubulin into microtubules are described in the examples. All five analogs inhibited the light scattering signal in a concentration-dependent manner, indicating that the noscapine analogs can bind to tubulin and inhibit microtubule assembly. Successful chemotherapy relies on the strategic induction of robust apoptosis in cancer cells while sparing normal cells. It is noteworthy that the benzofuranone noscapine analogs described herein did not affect the viability of normal human fibroblasts at concentrations as high as 100 μM.

Chemotherapeutic agents induce cell death by arresting cell cycle progression, upregulating the expression of pro-apoptotic molecules while downregulating survival signaling players that encumber apoptosis. The rate and extent to which cell lines from various tissue types respond to a particular test compound essentially depends on the status of death-resisting anti-apoptosis molecules, as well as death-favoring pro-apoptotic molecules in that cell type and how these molecules are affected upon drug administration. For example, survivin, an antiapoptotic protein of the inhibitor of apoptosis family that blocks apoptosis by inhibiting caspases has been shown to a player in dictating cellular sensitivity to 9-bromonoscapine. The data in the examples show that the compounds described herein are able to alter survivin levels as part of their anti-proliferative and pro-apoptotic program. Examining the expression levels of survivin upon treatment with these compounds caused a decline in survivin. Even though the sensitivity of various cancer cells to the compounds described herein may be cell-type dependent, it is apparent that tubulin presents a potential target for these compounds.

The compositions described herein contain an effective amount of the one or more noscapine analogs. The amount to be administered can be readily determined by the attending physician based on a variety of factors including, but not limited to, age of the patient, weight of the patient, disease or disorder to be treated, presence of a pre-existing condition, and dosage form to be administered (e.g., immediate release versus modified release dosage form). Typically, the effective amount is from about 0.1 mg/kg/day to about 200 mg/kg/day, more preferably from 0.1 mg/kg/day to 50 mg/kg/day, more preferably from 0.1 mg/kg/day to 25 mg/kg/day, and most preferably from 0.1 mg/kg/day to 10 mg/kg/day. Dosages greater or less than this may be administered depending on the diseases or disorder to be treated.

The compounds described herein can be administered to provide an effective amount to treat a variety of diseases and disorders including but not limited to, proliferative disorders (e.g., cancers), hypoxic ischemia in stroke patients, polycystic ovary disease, and amyotrophic lateral sclerosis (ALS).

A. Proliferative Disorders

1. Cancers

The noscapine analogs described herein can be administered to a subject in need thereof to treat the subject either prophylactically (i.e., to prevent cancer) or therapeutically (i.e., to treat cancer after it has been detected), including reducing tumor growth, reducing the risk of local invasiveness of a tumor, increasing survival time of the patient, and/or reducing the risk of metastasis of a primary tumor.

The compounds described herein can contact a target cell to inhibit the initiation and promotion of cancer, to kill cancer/malignant cells, to inhibit cell growth, to induce apoptosis, to inhibit metastasis, to decrease tumor size, to otherwise reverse or reduce the malignant phenotype of tumor cells, and combinations thereof. This may be achieved by contacting a tumor or tumor cell with a single composition or pharmacological formulation that includes the noscapine analog(s), or by contacting a tumor or tumor cell with more than one distinct composition or formulation, simultaneously, wherein one composition includes one or more noscapine analogs described herein and the other includes a second agent.

Exemplary cancers which can be treated include, but are not limited to, cancer of the skin, colon, uterine, ovarian, pancreatic, lung, bladder, breast, renal system, and prostate. Other cancers include, but are not limited to, cancers of the brain, liver, stomach, esophagus, head and neck, testicles, cervix, lymphatic system, larynx, esophagus, parotid, biliary tract, rectum, endometrium, kidney, and thyroid; including squamous cell carcinomas, adenocarcinomas, small cell carcinomas, gliomas, neuroblastomas, and the like. Assay methods for ascertaining the relative efficacy of the compounds described herein in treating the above types of cancers as well as other cancers are well known in the art.

The compounds described herein can also be used to treat metastatic cancer either in patients who have received prior chemo, radio, or biological therapy or in previously untreated patients. In one embodiment, the patient has received previous chemotherapy. Patients can be treated using a variety of routes of administration including systemic administration, such as intravenous administration or subcutaneous administration, oral administration or by intratumoral injection.

The noscapine analogs described herein can also be used to treat patients who have been rendered free of clinical disease by surgery, chemotherapy, and/or radiotherapy. In these aspects, the purpose of therapy is to prevent or reduce the likelihood of recurrent disease. Adjuvant therapy can be administered in the same regimen as described above to prevent recurrent disease.

### VI. Kits

In various aspects, a kit is envisioned containing one or more compounds described herein. The kit may contain one or more sealed containers, such as a vial, containing any of the compounds described herein and/or reagents for preparing a formulation or composition containing one or more of the compounds described herein. In some embodiments, the kit may also contain a suitable container means, which is a container that will not react with components of the kit, such as an eppendorf tube, an assay plate, a syringe, a bottle, or a tube. The container may be made from sterilizable materials such as plastic or glass.

The kit may further include instructions that outline the procedural steps for methods of treatment or prevention of disease, and will follow substantially the same procedures as described herein or are known to those of ordinary skill. The instruction information may be in a computer readable media containing machine-readable instructions that, when executed using a computer, cause the display of a real or virtual procedure of delivering a pharmaceutically effective amount of one or more compounds described herein.

### EXAMPLES

### Materials and Methods

In Silico Modeling Studies

Crystal structure coordinates of the tubulin heterodimer-colchicine models were used in the modeling studies. Theoretical binding sites of noscapinoids were generated by superimposing the drugs onto the colchicine molecule using Phase Flexible Ligand Superpositioning program from Schrodinger software and placing the resulting conformations into their respective 3D protein models. UCSF Chimera was used to determine hydrogen bonds and steric clashes of drugs docked to protein. Hydrophobic protein surface was made using Tripos Sybyl (v. 8.1) to further visualize sites of potential steric clashes.

Chemical Synthesis

All reactions were conducted in oven-dried (125° C.) glassware under nitrogen atmosphere. All common reagents and solvents were obtained from Sigma (St. Louis, Mo.) and used without further purification unless otherwise indicated. Solvents were dried by standard methods. The reactions were monitored by thin layer chromatography (TLC) using silica gel 60 F254 (Merck) pre-coated aluminum sheet. Flash chromatography was carried out on standard grade silica gel (230-400 mesh).

¹H NMR and ¹³C NMR spectra were measured in DMSO-d6 on a Bruker 400 NMR spectrometer. All proton NMR spectra were recorded at 400 MHz and were referenced with residual DMSO (2.50 ppm). Carbon NMR spectra were recorded at 100 MHz and were referenced with 77.27 ppm (CDCl₃) resonance of residual chloroform. Abbreviations for signal coupling are as follows: s, singlet; d, doublet; t, triplet; q, quartet; m, multiplet.

High resolution mass spectra were collected on Waters Q-TOF micro mass spectrophotometer using 3-nitrobenzyl alcohol, in some cases with addition of LiI as a matrix.

Cell Lines and Reagents

CEM (lymphoma), A549 (lung), PC-3 (prostate), MIA PaCa-2 (pancreatic), MCF-7 and MDA-MB-231 (breast) cancer cells were purchased from ATCC. PC3, A549, MDA-MB-231 and CEM were cultured in RPMI-1640 medium supplemented with 10% Fetal Bovine Serum (FBS) and 1% penicillin/streptomycin. MIA PaCa-2 and MCF-7 cells were cultured in DMEM supplemented with 10% FBS and 1% penicillin/streptomycin. Primary human dermal fibroblasts (HDF) from the dermis of normal human neonatal foreskin were obtained from the Dermatology Department, Emory University. MTT dye (Thiazolyl Blue Tetrazolium Bromide) dimethyl sulfoxide (DMSO), propidium iodide and RNase were purchased from Sigma (St. Louis, Mo.). Cells were cultured at 37° C. with 5% CO₂.

Tubulin Purification and Polymerization Assay

Microtubule proteins (MTP) consisting of ˜70% tubulin and ˜30% microtubule-associated proteins (MAPs) was isolated from bovine brain by three cycles of temperature-dependent polymerization and depolymerization. MAP-free tubulin (>99% pure) was purified from MTP by phosphocellulose chromatography. Purified tubulin was drop-frozen in liquid nitrogen, and stored at −80° C. until use.

The rate and extent of tubulin polymerization was monitored using a light scattering assay at 350 nm as described previously. Briefly, phosphocellulose-purified MAP-free tubulin (12-15 μM) was incubated with each compound at 0° C. for 10 mM in PEM buffer (80 mM PIPES, 3 mM MgCl₂, and 1 mM EGTA, pH 6.8) in a 96-well format. Following the addition of 1 mM GTP, assembly of tubulin was initiated by transferring the sample containing plate to Spectra Max Plus multi-well plate reader (Molecular Devices, USA). which was temperature pre-adjusted at 35° C.

Cytotoxicity Assay

MTT (3-(4,5-dimethylthiazol-2-yl)-2,5-diphenyltetrazolium bromide) assay was employed to evaluate the proliferative capacity of cells. Essentially, MTT is a colorimetric assay, which utilizes the colorless tetrazolium dye and converts it into a colored formazan salt, which can be quantified by measuring absorbance at 570 nm. Briefly, a 96-well format was used to seed 100 μA medium containing cells at a density of 5×10³ cellsper well. After 24 h of incubation, cells were treated with gradient concentration of the test compounds, which were dissolved in DMSO. The final concentration of DMSO in the culture medium was maintained at 0.1%. After 48 h of drug incubation, the spent medium was removed and the wells were washed twice with PBS. 100 μl of fresh medium and 10 μl of MTT (5 mg/ml in PBS) was added to the wells and cells were incubated at 37° C. in dark for 4 h. The formazan product was dissolved by adding 100 μl of 100% DMSO after removing the medium from each well. The absorbance was measured at 570 nm using a Spectra Max Plus multi-well plate reader (Molecular Devices, USA).

Cell-Cycle Analysis

Flow-cytometric evaluation of the cell-cycle status was performed as described previously. Control and drug treated cells were centrifuged, washed with ice-cold PBS, and fixed in 70% ethanol. Tubes containing the cell pellets were stored at 4° C. for at least 24 h. Cells were then centrifuged at 1000 g for 10 min and the supernatant was discarded. The pellets were washed twice with 5 ml of PBS and then stained with 0.5 ml of propidium iodide (0.1% in 0.6% Triton-X in PBS) and 0.5 ml of RNase A (2 mg/ml) for 45 min in dark. Samples were then analyzed on a BD FACSCanto II flow-cytometer (BD Biosciences, Sparks, Md.).

Immunoblot Analysis

Western blots were performed as described in the literature. Briefly, proteins were resolved by polyacrylamide gel-electrophoresis and transferred onto polyvinylidene difluoride membranes (Millipore). The membranes were blocked in Tris-buffered saline containing 0.05% Tween-20 and 5% fat-free dry milk and incubated first with primary antibodies against cleaved-PARP (Cell Signaling Inc., Beverly, Mass.) and survivin (Santa Cruz Biotechnology Inc., Santa Cruz, Calif.) and then with horseradish peroxidase-conjugated secondary antibodies (Santa Cruz Biotechnology Inc., Santa Cruz, Calif.). β-actin was from Sigma (St. Louis, Mo.). Specific proteins were visualized with enhanced chemiluminescence detection reagent according to the manufacturer's instructions (Pierce Biotechnology Inc., Rockford, Ill.).

Caspase 3/7 Activity Assay

Control or lysates of PC-3 cells treated with 25 μM noscapine analogs were tested for caspase-3-like activity using Ac-DEVD-7-amino-4-trifluoromethyl-coumarin, which detects the activities of caspase-3 and caspase-7 according to manufacturer's protocol (Calbiochem, San Diego, Calif.). The results were evaluated using Victor™ X5 multilabel reader (PerkinElmer, Inc., MA) and expressed as relative fluorescence units.

### Example 1

### In Silico Modeling Studies

Noscapine was discovered through a semi-rational structural screen of known microtubule poisons such as colchicine, podophyllotoxin, and MTC [2-methoxy-5-(2,3,4-trimethoxyphenyl)-2,4,6-cycloheptatrien-1-one], all of which are believed to bind to the same region of the cellular target, tubulin. The identification of noscapine was based on its structural resemblance with these drugs, such as a hydrophobic trimethoxyphenyl group and other hydrophobic domains (like a lactone, tropolone, or other aromatic rings) as well as small hydrophilic groups (like hydroxyl and amino groups). Since the 3.5 Å crystal structure of tubulin in complex with colchicine clearly shows the binding conformation of the drug, the structural similarity of noscapine (FIG. 2B) to colchicine (FIG. 2A) using a flexible ligand-superpositioning program was investigated. The overlap of two structures (FIG. 2C) yielded a geometric complementarity score of 72.75%, implying a strong structural similarity.

It is becoming recognizable that traditional docking approaches have several limitations when used with dynamic proteins such as tubulin. Thus, in order to model the binding of noscapine to tubulin, the space-coordinates of colchicine in its docked state were used to superimpose noscapine into the colchicine-binding domain of 1 SA0 PDB structure. Modeling data shows noscapine in the same position as colchicine, within the β-subunit near the intradimer interface. However, the methoxy group at position-7 showed multiple clashes with valine residue 315 in the β-subunit. This steric strain may be relieved upon O-demethylation at position-7 to yield a 7-hydroxy-compound. This may explain the observed increased activity of O-demethylated analogs that have been reported.

Given that replacing the methoxy group at position-7 with a smaller, hydroxyl group increased efficacy presumably by decreasing steric hindrance as discussed above, it has been suggested that substituting an increasingly large-sized group at this position would negatively impact the biological activity. In order to validate this predictive model, noscapine analogs were prepared by derivatizing position-7 on the benzofuranone ring system of noscapine with larger functional groups. When superimposed onto the colchicine-binding domain of the tubulin docking model, these analogs showed an increase in the number and magnitude of steric clashes at position-7 with an increase in the size of the substituted subgroup as shown in Table 2.

TABLE 2
Number and magnitude of steric clashes at position 7 of
the isobenzfuranone moiety

| | Average | Average | Average H- |
|---|---|---|---|
| Compound | Clashes | Overlap | Bonds |
| 3 | 32.8 | 1.017 | 2.6 |
| 4 | 52.6 | 1.131 | 2.2 |
| 5 | 62.6 | 1.091 | 2 |
| 6 | 61 | 1.163 | 2.4 |
| 7 | 56.2 | 1.1592 | 0.8 |

### Example 2

### Synthesis of (S)-7-hydroxy-6-methoxy-3-((R)-4-methoxy-6-methyl-5,6,7,8-tetrahydro[1,3]dioxolo-[4,5-g]isoquinolin-5-yl)isobenzofuran-1(3H)-one (2)

Noscapine (2.0 g, 4.84 mmol) was dissolved in anhydrous dimethyl formamide (DMF) (5.0 mL) followed by the addition of sodium azide (0.63 g, 9.68 mmol) and sodium iodide (0.36 g, 2.42 mmol). The mixture was stirred vigorously at 140° C. for 4 h. The mixture was concentrated under reduced pressure to yield a dark residue which was dissolved in EtOAc (50 mL). The insoluble material was filtered through celite and the filtrate was diluted with ethyl acetate (EtOAc) (150 mL) followed by washing with water (2×25 mL) and brine (2×25 mL). The organic layer was dried over sodium sulfate and concentrated under reduced pressure to give crude product which was crystallized from methanol. The product 2 was isolated as off-white needles. (78% yield): mp 142-143° C.; ¹H NMR (DMSO-d6, 400 MHz): δ 9.73 (s, 1H), 7.11 (d, J=8.0 Hz, 1H), 6.47 (s, 1H), 6.01 (m, 2H), 5.81 (d, J=8.0 Hz, 1H), 5.48 (d, J=4.0 Hz, 1H), 4.24 (d, J=4.0 Hz, 1H), 3.96 (s, 3H), 3.79 (s, 3H), 2.48-2.34 (m, 2H) 2.43 (s, 3H), 2.31-2.18 (m, 1H), 1.95-1.83 (m, 1H): ¹³C NMR (CDCl₃, 100 MHz): δ 174.6, 161.9, 151.6, 148.1, 140.7, 140.3, 133.9, 131.6, 116.8, 113.9, 111.8, 102.7, 102.4, 100.6, 80.9, 60.7, 59.3, 55.4, 48.2, 45.0, 25.76: HRMS: [M+H]⁺ [C₂₁H₂₁NO₇+H], calcd: 400.1396. found: 400.1382.

### Example 3

### Synthesis of (S)-5-methoxy-1-(R)-4-methoxy-6-methyl-5,6,7,8-tetrahydro-[1,3]dioxolo[4,5-g]isoquinolin-5-yl)-3-oxo-1,3-dihydroisobenzofuran-4-yl acetate (3)

Compound 2 (0.2 g, 0.5 mmol), was dissolved in anhydrous tetrahydrofuran (THF) (5.0 mL) followed by the addition of acetic anhydride (57 μL, 0.6 mmol) and dimethylamino pyridine (12 mg, 0.1 mmol). The mixture was stirred at ambient temperature for 4 h, the reaction progress was monitored by TLC, solvent was removed in vacuo and the residue thus obtained was dissolved in EtOAc (25 mL) followed by washing with water (2×25 mL). The organic layer was dried over sodium sulfate and concentrated under reduced pressure to give crude product which was separated over flash silica using methanol in chloroform as eluent (1:99) to afford compound 3 which was crystallized using methanol to yield pinkish crystals. (76% yield): mp 111-113° C.; ¹H NMR (DMSO-d₆, 400 MHz): δ 7.40 (d, J=8.4 Hz, 1H), 6.48 (s, 1H), 6.40 (d, J=8.4 Hz, 1H), 6.01 (s, 2H), 5.62 (d, J=4.4 Hz, 1H), 4.25 (d, J=4.4 Hz, 1H), 3.95 (s, 3H), 3.81 (s, 3H), 2.67-2.42 (m, 3H), 2.42 (s, 3H) 2.32 (s, 3H), 1.95-1.85 (m, 1H); ¹³C NMR (DMSO-d₆, 100 MHz): δ 168.5, 167.2, 151.7, 148.6, 140.5, 136.0, 134.3, 131.7, 121.4, 120.5, 119.5, 102.9, 101.3, 81.8, 60.8, 59.7, 57.1, 49.3, 45.9, 27.2, 20.6: HRMS: [M+H]⁺ [C₂₃H₂₂NO₈+H]⁺, calcd: 442.1502. found: 442.1505.

### Example 4

### Synthesis of (S)-5-methoxy-1-(R)-4-methoxy-6-methyl-5,6,7,8-tetrahydro-[1,3]dioxolo[4,5-g]isoquinolin-5-yl)-3-oxo-1,3-dihydroisobenzofuran-4-yl benzoate (4)

Compound 2 (0.2 g, 0.5 mmol), was dissolved in anhydrous THF (5 mL), potassium carbonate (0.1 g) was added and the mixture was cooled over an ice bath (0-4° C.). Benzoyl chloride (76 μl, 0.65 mmol) was added drop-wise and stirred vigorously at 0° C. then warmed to room temperature (RT) overnight. Solvent was removed in vacuo and the residue thus obtained was dissolved in ethyl acetate (25 mL) and washed with water (2×25 mL). The combined organic layers were dried over sodium sulfate and concentrated under reduced pressure to give crude product which was separated over flash silica using methanol in chloroform as eluent (1:99) to obtain compound 4 which was crystallized with methanol to yield dark yellow crystals. (92% yield): mp 152° C.; NMR (DMSO-d₆, 400 MHz): δ 8.14 (m, 2H), 7.79 (m, 1H), 6.40 (d, J=8.4 Hz, 1H), 7.64 (m, 2H), 7.47 (d, J=8.4 Hz, 1H), 6.50 (s, 1H), 6.44 (bs, 1H), 6.02 (s, 2H), 5.67 (d, J=4.4 Hz, 1H), 4.29 (d, J=4.4 Hz, 1H), 3.98 (s, 3H), 3.82 (s, 3H) 2.62 (m, 1H), 2.54 (m, 1H) 2.44 (s, 1H), 2.33 (m, 1H), 1.93 (m, 1H); ¹³C NMR (CDCl₃, 100 MHz), δ 170.6, 166.7, 164.2, 152.1, 149.2, 140.4, 140.1, 136.9, 133.9, 132.7, 131.4, 130.5, 129.9, 128.9, 128.3, 121.5, 120.4, 118.5, 102.4, 100.9, 81.0, 61.2, 59.0, 56.9, 47.8, 45.0, 25.2.: HRMS: [M+H]⁺ [C₂₈H₂₅NO₈+H]⁺, calcd: 504.1658. found: 504.1668.

### Example 5

### General Synthesis Procedure for Carbamate Analogs 5-7

Compound 2 (0.25 g, 0.626 mmol), was dissolved in anhydrous dichloromethane (5 mL) followed by the addition of ethyl isocyanate (54 μl, 0.689 mmol) and the dimethylamino pyridine (8 mg, 0.065 mmol). The mixture was stirred vigorously at ambient temperature for 4 h. The mixture was condensed under reduced pressure to dryness. The residue was dissolved in ethyl acetate (25 mL) and washed with water (2×10 mL). The organic layer was dried over sodium sulfate and concentrated under reduced pressure to give crude product which was chromatographed over flash silica using methanol in chloroform as eluent (2:98) to yield carbamate analogs 5-7.

### (S)-5-methoxy-1-0)-4-methoxy-6-methyl-5,6,7,8-tetrahydro-[1,3]dioxolo[4,5-g]isoquinolin-5-yl)-3-oxo-1,3-dihydroisobenzofuran-4-yl phenylcarbamate (6)

Yellow solid. (64% yield): mp 133-135° C.; ¹H NMR (DMSO-d₆, 400 MHz): δ 7.81 (t, J=5.2 Hz, 1H), 7.34 (d, J=8.4 Hz, 1H), 6.48 (s, 1H), 6.35 (d, J=8.4 Hz, 1H), 6.00 (s, 2H), 5.58 (d, J=4.4 Hz, 1H), 4.25 (d, J=4.4 Hz, 1H), 3.95 (s, 3H), 3.79 (s, 3H), 3.09 (q, J=7.2 Hz, 2H), 2.67-2.42 (m, 3H), 2.43 (s, 3H), 1.94-1.92 (m, 1H), 1.09 (t, J=7.2 Hz, 3H); ¹³C NMR (DMSO-d₆, 100 MHz): δ 167.2, 153.4, 152.5, 148.6, 140.6, 137.2, 134.4, 131.8, 121.2, 120.5, 119.2, 116.9, 102.9, 101.3, 81.6, 79.7, 60.9, 59.7, 57.04, 45.89, 49.3, 35.9, 27.1, 15.3: HRMS: [M+H]⁺ [C₂₄H₂₆N₂O₈+H]⁺, calcd: 471.1767. found: 471.1761.

### (S)-5-methoxy-1-((R)-4-methoxy-6-methyl-5,6,7,8-tetrahydro-[1,3]dioxolo[4,5-g]isoquinolin-5-yl)-3-oxo-1,3-dihydroisobenzofuran-4-yl benzylcarbamate (7)

Yellow solid. (78% yield): mp 122-123° C.; ¹H NMR (DMSO-d₆, 400 MHz): δ 8.39 (t, 5.2 Hz, 1H), 7.38-7.27 (m, 6H), 6.48 (s, 1H), 6.37 (d, J=8.4 Hz, 1H), 6.01 (s, 2H), 5.60 (d, J=4.4 Hz, 1H), 4.31-4.27 (m, 2H), 4.24 (d, J=4.4 Hz, 1H), 3.95 (s, 3H), 3.81 (s, 3H), 2.72-2.48 (m, 2H), 2.44 (s, 3H), 2.32-2.28 (m, 1H), 1.91-1.86 (m, 1H); ¹³C NMR (DMSO-d₆, 100 MHz): δ 158.6, 154.1, 152.5, 148.6, 140.6, 137.2, 134.4, 131.8, 121.2, 120.5, 119.2, 116.9, 102.9, 101.3, 81.6, 79.7, 60.9, 59.7, 46.4, 35.9, 27.1: HRMS: [M+H]⁺ [C₂₉H₂₈N₂O₈+H]⁺, calcd: 533.1924. found: 533.1926.

### Example 6

### Benzofuranone Ring Substituted Noscapine Analogs Inhibit Tubulin Polymerization In Vitro

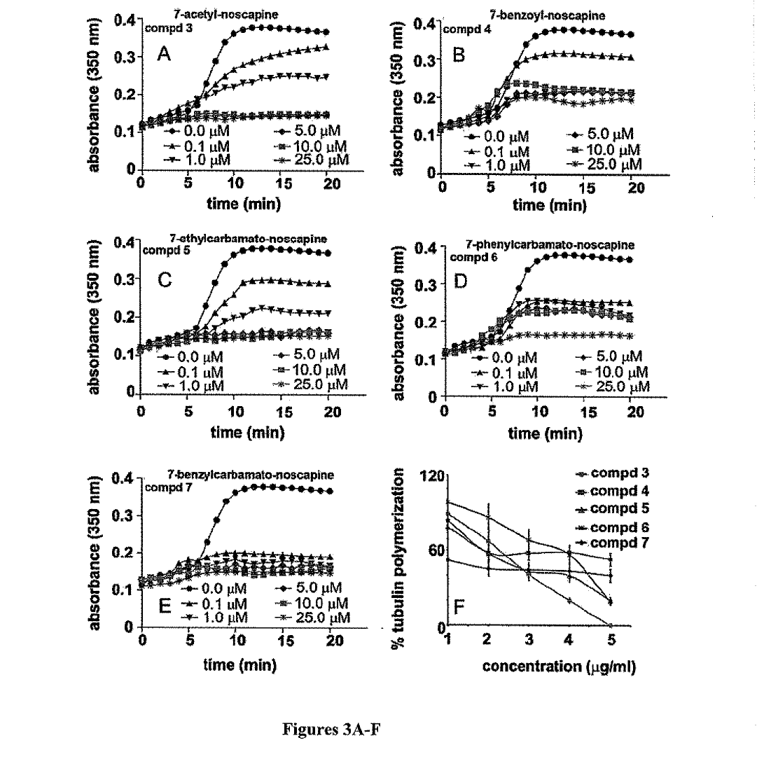

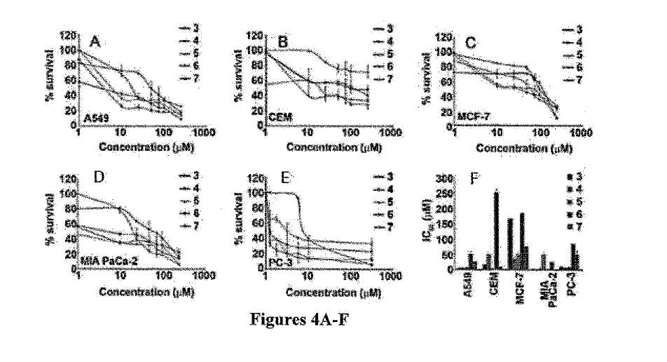

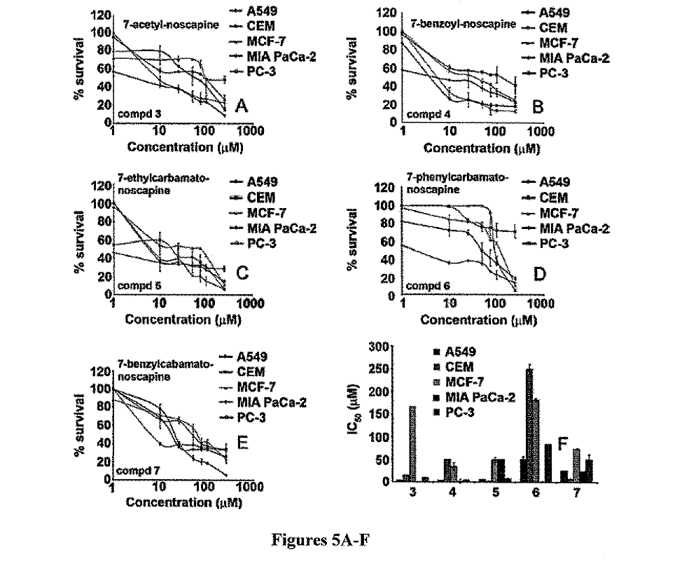

To determine the anti-tubulin activity of these benzofuranone ring substituted noscapine analogs, their effects on tubulin polymerization were examined in vitro using the assay described above. The effects of various concentrations of all five noscapine analogs on the polymerization of tubulin into microtubules are shown in FIG. 3. All five analogs inhibited the light scattering signal in a concentration-dependent manner, indicating that these benzofuranone ring substituted noscapine analogs can bind to tubulin and inhibit microtubule assembly (see FIGS. 3A-3E). The IC₅₀ values for compounds 3-7 are shown in FIG. 3F.

### Example 7

### Benzofuranone Ring Substituted Noscapine Analogs Display Significant Antiproliferative Activity

The newly synthesized 7-position analogs of noscapine were tested for their antiproliferative activity in various cancer cells using the MTT assay as described above. FIG. 4 shows line plots of cell survival versus gradient concentrations of various compounds to yield IC₅₀ values of each analog in different cell lines (see FIGS. 4A-4E). The IC₅₀ values for compounds 3-7 are shown in FIG. 4F.

The IC₅₀ value (drug concentration at which 50% inhibition of cell proliferation occurs) of these synthesized compounds are presented in Table 3.

TABLE 3
In vitro toxicity (IC₅₀, μM) of noscapine analogs

| | | Cancer cell line, IC₅₀ (μM) | | | | |
|---|---|---|---|---|---|---|
| Compound | R | A549 | CEM | MCF-7 | MIA PaCa-2 | PC-3 |
| 3 | —CH₃ | 3.2 | 15.5 | 166.0 | 1.0 | 9.3 |
| 4 | —Ph | 4.5 | 49.0 | 34.0 | 1.7 | 4.8 |
| 5 | —NHEt | 5.6 | 1.7 | 49.0 | 49.0 | 6.6 |
| 6 | —NHPh | 50.0 | 250.0 | 182.0 | 1.0 | 83.2 |
| 7 | —NHCH₂Ph | 25.0 | 7.1 | 74.1 | 24.5 | 49.0 |
| Noscapine | | 73.0 | 20.0 | 45.0 | 70.0 | 51.0 |

To appreciate the potency of these novel 7-position substituted benzofurananone noscapine analogs, the IC₅₀ values of the parent molecule noscapine for the various cell lines under study are indicated in Table 3 (bottom-row).

Among the noscapine analogs tested, compound 3 (7-acetyl-noscapine) was observed to be generally most-effective against most cell lines used in the study (FIGS. 4A-E) except for breast cancer cells (FIG. 4C). Pancreatic cancer MIA PaCa-2 cells were particularly sensitive to compounds 3, 4 and 6 (IC₅₀ in the range of 1 to 1.7 μM) as compared to compounds 5 and 7 (IC₅₀ 49.0 and 24.5 respectively) (FIG. 4D and Table 3). Lung cancer A549 cells also showed low IC₅₀ for compounds 3, 4 and 5 (Table 3) compared to CEM lymphoma cells, which were observed to be more sensitive towards compound 5 (FIG. 4B). The IC₅₀ values of the cell lines, A549, CEM, MIA PaCa-2 and PC-3 were within 10 μM (Table 3) for compounds 3, 4 and 5. MCF-7, with higher IC₅₀ values was found to be resistant towards these analogs. FIG. 4F is a bar-graph representation depicting a comparison of the IC₅₀ values of five noscapine analogs towards each cancer cell used in the study.

As the bulk of the substituent increased in compound 4 (7-benzoyl-noscapine), an increase in IC₅₀ values was observed, which was even more pronounced with compound 5 and 6, with a few exceptions. The majority of the cell lines were relatively resistant towards compound 6 (with IC₅₀ values ranging from 50-250 μM, Table 3). The significantly higher IC₅₀ values of compound 6 indicated that the bulk of the substituent plays an important role in determining the biological activity in cellular systems. Compound 7, as observed from Table 3, showed relatively lower IC₅₀ values when compared to compound 6 (Table 3). It is reasonable to speculate that the presence of a CH₂ group between the nitrogen atom and aromatic ring system provides flexibility to compound 7, which perhaps relates to its higher activity. Comparison with noscapine demonstrated that 7-position analogs, in particular, compounds 3-5, are significantly better in antiproliferative activity (Table 3).

### Example 8

### Benzofuranone Ring Substituted Noscapine Analogs Show Significant Inter-Line Variation

The expression level of oncogenes, tumor suppressor, and key molecules that regulate apoptosis, drug-resistance and angiogenesis can affect the sensitivity of tumor cells towards any given anti-cancer agent. It is recognizable that anti-tubulin agents like paclitaxel and docetaxel offer superior anti-tumor outcomes in solid malignancies such as breast and ovarian, while hematological malignancies are typically best managed by vinca alkaloids (vinblastine, vincristine etc.), Differential sensitivity of cancer cell lines was observed for each noscapine analog suggesting significant inter-line variations (FIG. 5). Most cancer cells (lung, lymphoma, pancreatic and prostate) significantly responded to compound 3 and displayed much lower IC₅₀ values in the range of 1-15 μM (FIG. 5A). However, a very high IC₅₀ (166.0 μM) was observed for the breast cancer cell line, MCF-7 (Table 3, FIG. 5A), suggesting a high degree of inter-line variability. Compound 4 with a COPh (benzoyl) substituent, although quite effective against lung, pancreatic and prostate cancer cells (IC₅₀ values less than 5 μM) showed resistance towards lymphoma (IC₅₀ 49.0 μM) and breast cancer (IC₅₀ 34.0 μM) cells (FIG. 7B). On the other hand, compound 5 showed lower activity in CEM (1.7 μM), A549 (5.6 μM) and PC-3 (6.6 μM) (FIG. 5C and Table 3). Interestingly, pancreatic cancer cells were very sensitive to compound 6 (IC₅₀=1 μM, FIG. 5D). These differences in cellular sensitivities to the same compound may be due to altered expression of β-tubulin isotypes, or point mutations in tubulin resulting in alterations of expression patterns of post-translational modifications of tubulin regulatory proteins, such as stathmin, microtubule associated protein (MAP), tau and MAP4. These changes in microtubule accessory proteins have been well recognized to affect microtubule dynamicity and can perhaps contribute to the development of drug resistance.

Interestingly, compounds 3-7 did not inhibit cell proliferation in normal human dermal fibroblasts (HDFs) even at concentrations as high as 100 μM. The proliferation capacity of these compounds was compared with the parent compound noscapine and the 9-position substituted analog, 9-bromonoscapine.

### Example 9

### Benzofuranone Ring Substituted Noscapine Analogs Cause Cell Cycle Arrest Followed by Apoptosis

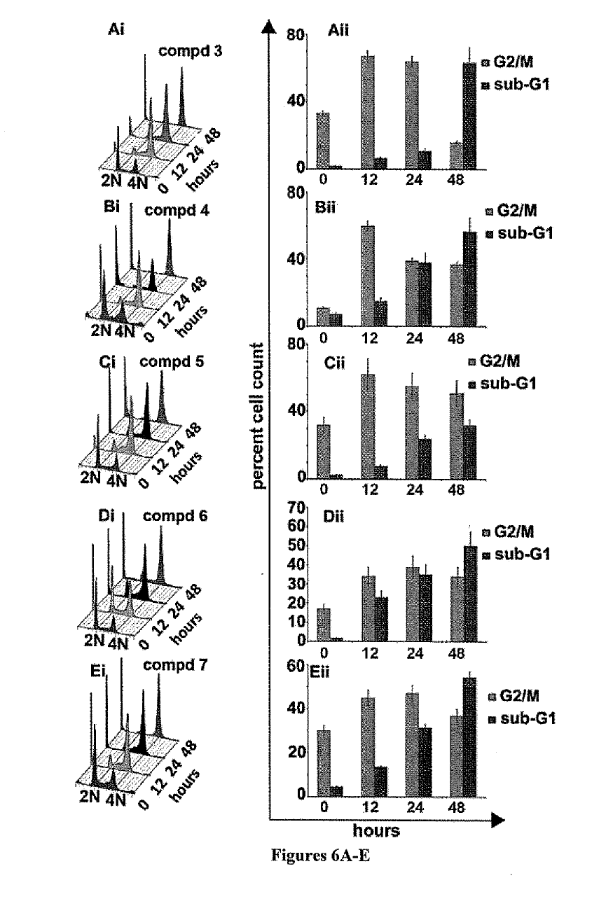

To gain further insights into the precise mechanisms responsible for inhibition of cellular proliferation, we next examined the effect of benzofuranone ring substituted analogs on cell cycle distribution profiles of breast cancer MDA-MB-231 cells. The effect of compounds 3-7 on mitotic index (percent G2/M cells) and apoptotic index (percent sub-G1 cells) as a function of time in MDA-MB-231 cells was studied using fluorescence activated cell sorting (FACS) analysis. All 5 compounds were used at 25 μM concentration over 48 h of treatment. FIG. 6 (Ai-Ei) shows cell-cycle profiles upon treatment with compounds 3-7 over various time points (0, 12, 24, and 48 h) in a three-dimensional disposition. While 2N and 4N DNA complements are representative of G1 and G2/M populations, respectively, sub-G1 hypodiploid population is usually suggestive of fragmented DNA and is a hallmark of apoptosis.

Treatment of MDA-MB-231 cells with compounds 3-7 showed significant accumulation of cells in G2/M phase until 24 h of drug exposure (FIG. 6 Aii-Eli). The G2/M population however started to decline beyond 24 h and thereafter a concomitant increase of sub-G1 population was observed until 48 h. In case of compounds 3 and 5, as is evident from the bar graph; the G2/M population increased considerably at 12 and 24 h (FIG. 6 Aii and Cii). However, at 48 h the G2/M population decreased significantly perhaps leading to apoptotic cell death in case of compound 3 (FIG. 6 Aii). Even after 48 h, compound 5 treated cells mostly remained arrested in G2/M phase and the apoptotic index was lower compared to compound 3. Compounds 4, 6 and 7 also show similar pattern of G2/M arrest over 24 h of treatment followed by a decline in the percent G2/M cells and emergence of a hypodiploid population, indicative of apoptosis (FIG. 6 Bii, Dii, and Eii).

Although staining of DNA with propidium iodide is extremely useful to determine the percent cell population in various cell-cycle phases, it has its limitations. For example, it cannot dissect out the differences between G2 and M phases as both have 4N DNA amounts. Thus, to explore further, a mitosis specific marker, MPM-2, was used to distinctly identify which phase the cells were in. The data revealed that all five noscapine analogs appeared to induce strong G2 arrest starting as early as 12 h (˜65%) continuing till 24 h. In case of compound 5, the arrest was maintained until 48 h. Cell population corresponding to the G2/M peak was clearly negative for MPM2, suggesting that the cells accumulated in G2 phase for a long time before succumbing to apoptosis.

In addition, compounds 3-7 induced apoptotic cell death in PC-3 cells, which was associated with decreased expression of the anti-apoptotic protein surviving, an enhanced caspase-3 activity, and cleavage of PARP (Supple FIG. 3A).

1. A compound of Formula I: 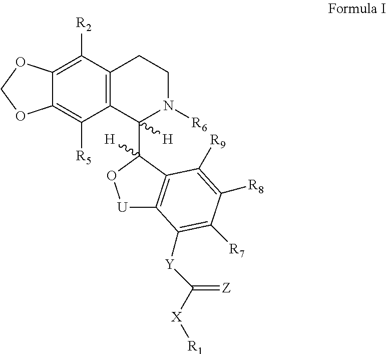
wherein,
R₁ is selected from hydrogen; substituted or unsubstituted alkyl, alkenyl, alkynyl, cycloalkyl, heterocycloalkyl, cycloalkenyl, heterocycloalkenyl, aryl, and heteroaryl;
X and Y are independently absent or S, O, and NR₃, wherein R₃ is H, alkyl, substituted or unsubstituted alkyl, alkenyl, alkynyl, cycloalkyl, heterocycloalkyl, cycloalkenyl, heterocycloalkenyl, aryl, and heteroaryl;
Z is O, S, or N;
R₂, R₅, and R₇-R₉ are independently selected from hydrogen; halogen, substituted or unsubstituted alkyl, alkenyl, alkynyl, cycloalkyl, heterocycloalkyl, cycloalkenyl, heterocycloalkenyl, aryl, heteroaryl, alkylaryl, alkylheteroaryl, arylalkyl, or heteroarylalkyl; —OR′; —NR′R″; —(CH₂)ₘNR′R″, wherein m is 0, 1, or 2; —NO₂; —CF₃; —CN; —C₂R′; —SR′; —N₃; —C(═O)NR′R″; —NR′C(═O)R″; —C(═O)R′; —C(═O)OR′; —OC(═O)R′; —O(CR′R″)ᵣC(═O)R′; —O(CR′R″)ᵣNR″C(═O)R′; —O(CR′R″)ᵣNR″SO₂R′; —OC(═O)NR′R″; —NR′C(═O)OR″; —SO₂R′; —SO₂NR′R″; and —NR′SO₂R″; wherein R′ and R″ are individually hydrogen or substituted or unsubstituted alkyl, alkenyl, alkynyl, cycloalkyl, heterocyclyl, cycloalkenyl, heterocycloalkenyl, aryl, heteroaryl, arylalkyl, heteroalkyls, alkylaryl, alkylheteroaryl, and r is an integer from 1 to 6;
R₆ is hydrogen or substituted or unsubstituted alkyl, alkenyl, alkynyl, cycloalkyl, heterocycloalkyl, cycloalkenyl, heterocycloalkenyl, aryl, heteroaryl, arylalkyl, heteroarylalkyl, alkylaryl, alkylheteroaryl, —C(═W)NR′R″, wherein W is O, S, or N and R′ and R″ are as defined above; and
U is CH₂; C═O; C═S; C═NH; C═NR, wherein R is as defined above for R′ and R″; CHOH or CHOR, wherein R is as defined above for R′ and R″; or CR₁₀R₁₁, wherein R₁₀ and R₁₁ are as defined above for R₂.

2. The compound of claim 1, wherein X is absent.

3. The compound of claim 2, wherein R₁ is methyl

4. The compound of claim 2, wherein R₁ is phenyl.

5. The compound of claim 3, wherein R₂ is hydrogen, R₅ is methoxy, and R₇ is methyl.

6. The compound of claim 4, wherein R₂ is hydrogen, R₅ is methoxy, and R₇ is methyl.

7. The compound of claim 1, wherein X is NR₃.

8. The compound of claim 7, wherein R₃ is hydrogen.

9. The compound of claim 8, wherein R₁ is ethyl.

10. The compound of claim 8, wherein R₁ is phenyl.

11. The compound of claim 8, wherein R₁ is benzyl.

12. The compound of claim 9, wherein R₂ is hydrogen, R₅ is methoxy, and R₇ is methyl.

13. The compound of claim 10, wherein R₂ is hydrogen, R₅ is methoxy, and R₇ is methyl.

14. The compound of claim 11, wherein R₂ is hydrogen, R₅ is methoxy, and R₇ is methyl.

15. A compound of Formula II 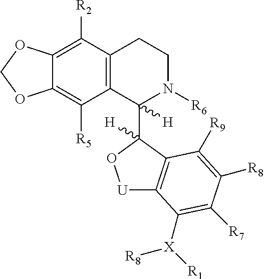
wherein R₂, R₅-R₉, and U are as defined above, X is N, S, or O, and R₁ and R₈ are absent or, as valence allows, independently selected from hydrogen; substituted or unsubstituted alkyl, alkenyl, alkynyl, cycloalkyl, heterocycloalkyl, cycloalkenyl, heterocycloalkenyl, aryl, and heteroaryl.

16. A pharmaceutical composition comprising the compound of claim 1 and a pharmaceutically acceptable carrier.

17. A method of treating a proliferative disease, the method comprising administering to a patient in need thereof an effective amount of the compound of claim 1.

18. The method of claim 17, wherein the compound is administered parenterally.

19. The method of claim 17, wherein the compound is administered enterally.

20. The method of claim 17, wherein the proliferative disease is a cancer.

21. A pharmaceutical composition comprising the compound of claim 15 and a pharmaceutically acceptable carrier.

22. A method of treating a proliferative disease, the method comprising administering to a patient in need thereof an effective amount of the compound of claim 2.

23. The method of claim 22, wherein the compound is administered parenterally.

24. The method of claim 22, wherein the compound is administered enterally.

25. The method of claim 22, wherein the proliferative disease is a cancer.
